(19) United States
(12) Patent Application Publication
(10) Pub. No.: US 2013/0089495 A1
(43) Pub. Date: Apr. 11, 2013

(54) METHOD FOR SCREENING SIZE OF CARRIER

(71) Applicants: Patrick C.H. Hsieh, Tainan City (TW); Hui-Jing Li, Kaohsiung City (TW)

(72) Inventors: Patrick C.H. Hsieh, Tainan City (TW); Hui-Jing Li, Kaohsiung City (TW)

(73) Assignee: National Cheng Kung University, Tainan City (TW)

(21) Appl. No.: 13/465,150

(22) Filed: May 7, 2012

Related U.S. Application Data
(60) Provisional application No. 61/543,400, filed on Oct. 5, 2011.

(51) Int. Cl. A61K 49/00; A61K 51/00; B82Y 5/00; B82Y 15/00

(52) U.S. Cl. 424/1.11; 424/9.1; 424/9.6; 977/773; 977/906; 977/927

(57) ABSTRACT

The present invention provides a method for screening the size of carrier for a subject in need, comprising: (a) providing a series of labeled carriers which have different sizes; (b) administering one of the series of carriers to a subject who suffers from an organ dysfunction; (c) monitoring biodistribution of the carrier of step (b) in said subject; (d) repeating steps (b) and (c) until all the series of carriers are administered and all the biodistribution of the series of carriers are monitored; and (e) determining the size of carrier for said subject in accordance with the retention time of the series of carriers in the dysfunctional organ of said subject. The method can be used as a screening platform for drug carrier, in which the optimal size of carrier can be screened for the dysfunctional organ of the subject.

### BACKGROUND OF THE INVENTION

1. Field of the Invention

The present invention provides a method for screening the size of carrier for a subject in need.

2. Description of the Related Art

The convergence of nanotechnology and biomaterials has spawned nanoparticles^{1,2}, which have been widely used in medical applications, including drug delivery^{3,4}, tissue engineering^{5,6}, and medical imagine^{7,8}. Accordingly, the toxicity of nanoparticles must be fully characterized before any nanoscale system can be used safely and efficiently for medical applications. There has been an increase in the number of studies reporting that the physical and chemical properties of size, shape, surface charge and functional groups influence the biodistribution, accumulation, and excretion of nanoparticles^{9,10}. In previous studies, investigators have controlled the size and shape of nanoparticles to manipulate their behavior and to achieve enhanced, targeted drug delivery^{11,12}. Research has also demonstrated that particle size greatly affects the transport and fate of the particle itself^{13,14}. However, a comprehensive and systematic evaluation of nanoparticle on their biodistribution and on biological host responses in a quantitative and unambiguous manner has not yet been published. In the present invention, we develop methods to characterize the size effect of nanoparticles in vivo and to study the biodistribution of these particles in clinically relevant models.

### SUMMARY OF THE INVENTION

Accordingly, the invention provides a method for detecting nanoparticles (NPs) in vivo retention (such as by HPLC), and the NPs size-dependent distribution in different conditions in various organs.

One object of the present invention is to provide a method for screening the size of carrier for a subject in need, which can be used as a screening platform for drug carrier, in which the optimal size of carrier can be screened for the organ and/or tissue affected by the condition of the subject. To achieve these objectives, the present invention provides a method for screening the size of carrier for a subject in need, comprising: (a) providing a series of labeled carriers which have different sizes; (b) administering one of the series of carriers to a subject who suffers from a condition selected from organ dysfunction, inflammation, cancer formation or other injured or abnormal conditions; (c) monitoring biodistribution of the carrier of step (b) in said subject; (d) repeating steps (b) and (c) until all the series of carriers are administered and all the biodistribution of the series of carriers are monitored; and (e) determining the size of carrier for said subject in accordance with the retention amount of the series of carriers in the tissue and/or organ affected by the condition of said subject.

In a preferred embodiment, the series of carriers are composed of an organic or an inorganic material, such as polystyrene, poly(lactic-co-glycolic acid) (PLGA), polylactic acid (PLA), polyglycolic acid (PGA), gelatin, fibrin, agarose, chitosan, liposome, hyaluronic acid (HA), poly (ethylene glycol) (PEG), poly(propylacrylic acid) (PPAA) and N-isopropylacrylamide (NIPAAm); or composed of a metal material, such as gold.

In a preferred embodiment, the series of carriers are nanoparticles having a size in the range of 0.1-1000 nm; more preferably, in the range of 1-500 nm; even more preferably, in the range of 20-500 nm.

In a preferred embodiment, each of the series of carriers is fluorescence-labeled, radio-labeled, iron oxide-loaded, labeled by other materials or by methods for detection of the nanoparticles.

In a preferred embodiment, each of the series of carriers is administered by systemic intravascular, intramuscular or subcutaneous injection, oral intake, inhalation, or local skin, anal or vaginal administration.

In a preferred embodiment, the biodistribution of each carrier is monitored through in vivo, ex vivo or in vitro imaging system; more preferably, the imaging system comprises bioluminescence images (including immunofluorescent imaging), X-ray, CT, MRI, NMR, HPLC, PET/SPECT, ultrasound, OCT or other imaging methods for detecting the carriers, particularly, the nanoparticles.

In a preferred embodiment, the organ affected by the condition is brain, and the size of carrier is in the range of 0.1-1000 nm; more preferably, in the range of 1-500 nm; even more preferably, in the range of 20-500 nm. In the case of mammals, particularly in mice, the best size of carrier is in the range of less than 100 nm.

In a preferred embodiment, the organ affected by the condition is skin, and the size of carrier is in the range of 0.1-1000 nm; more preferably, in the range of 1-500 nm; even more preferably, in the range of 20-500 nm. In the case of mammals, particularly in mice, the best size of carrier is in the range of less than 100 nm.

In a preferred embodiment, the tissue affected by the condition is muscle, and the size of carrier is in the range of 0.1-1000 nm; more preferably, in the range of 1-500 nm; even more preferably, in the range of 20-500 nm. In the case of mammals, particularly in mice, the best size of carrier is in the range of less than 100 nm.

In a preferred embodiment, the organ affected by the condition is liver or spleen, and the size of carrier is in the range of 0.1-1000 nm; more preferably, in the range of 1-500 nm; even more preferably, in the range of 20-500 nm. In the case of mammals, particularly in mice, the best size of carrier is 20-500 nm.

In a preferred embodiment, the organ affected by the condition is lung, and the size of carrier is in the range of 0.1-1000 nm; more preferably, in the range of 1-500; even more preferably, in the range of 20-500 nm. In the case of mammals, particularly in mice, the best size of carrier is in the range of larger than 100 nm.

In a preferred embodiment, the organ affected by the condition is kidney, and the size of carrier is in the range of 0.1-1000 nm; more preferably, in the range of 1-500 nm; even more preferably, in the range of 20-500 nm. In the case of mammals, particularly in mice, the best size of carrier is in the range of larger than 20 nm.

### BRIEF DESCRIPTION OF THE DRAWINGS

FIG. 1: Nanoparticle fluorescein extraction and efficiency. (a) TEM images of 20, 50, 100, 200 and 500 nm nanoparticles. Scale bar: 100 nm. (b) Flow chart of nanoparticle fluorescein injection and extraction. (c) Standard curve fittings from known concentrations of 20, 50, 100, 200 and 500 nm nanoparticle solutions analyzed by HPLC. (d) Fluorescence intensity of fluorescein extracted by o-xylene only, o-xylene combined with 1 M KOH or o-xylene after 1 M KOH digestion in a 60° C. oven overnight (black, red, and blue dashed lines, respectively).

FIG. 2: The biodistribution of nanoparticles of different sizes following systemic injection into normal mice. (a) Nanoparticle biodistribution in the vital organs, including the brain, heart, lungs, liver, spleen and kidneys imaged ex vivo by an in vivo imaging system (IVIS). (b) Total nanoparticle retention by organ of the six vital organs and the blood. (c) The sum of the total nanoparticle retention in the six vital organs by nanoparticle size. (d) IVIS images of nanoparticle retention in the peripheral tissues and urine.

FIG. 3: The biodistribution of nanoparticles of different sizes in mice treated with bacterial lipopolysaccharide (LPS). (a) IVIS images of the nanoparticle biodistribution in the six vital organs. (b) Total nanoparticle retention by organ in the six vital organs and the blood. (c) Total nanoparticle retention in the six vital organs by nanoparticle size compared with FIG. 1E.*, p<0.05; **, p<0.01 by Student's t test. (d) IVIS images of nanoparticle retention in peripheral tissues and urine.

FIG. 4: The tissue retention of nanoparticles of different sizes in the major vital organs of a mouse following systemic injection. Immunofluorescent staining of tissue sections showing a size effect of nanoparticle retention in the (a) brain , (b) lungs , (c) liver and (d) spleen under normal and inflammatory conditions. Red, nanoparticles; green, isolectin; blue, DAPI. Scale bar: 50 μm.

FIG. 5: The size effect of poly(lactic-co-glycolic-acid) nanoparticle retention in mouse muscles after hindlimb ischemia. (a) HPLC quantification of different-sized fluorescent polystyrene nanoparticles that were administrated to mice after 6 hours, 1 day, or 3 days of reperfusion. Blood flow measurements were first normalized to nonischemic measurements (n≧4 in each group). (b) Immunofluorescent staining of tissue sections showing nanoparticle retention in nonischemic and ischemic muscles after reperfusion for 1 hour. Scale bar: 100 μm. (c) Fabrication process and TEM images of PLGA and PLGA conjugated to quantum dots (PLGA-QD). PEI, polyethylenimine. Scale bar: 100 nm. (d) In vivo and ex vivo fluorescence images from IVIS of PLGA or PLGA-QD-injected hindlimbs of ischemic mice subjected to 1 day of reperfusion. Top panel, in vivo image; yellow arrow, ischemic leg; bottom panel, ex viva muscle image. (e) Fluorescence quantification of ex vivo images in (d) normalized to nonischemic muscle fluorescence (n≧4 in each group). *, p<0.05; **, p<0.01 by Student's t-test.

FIG. 6: Cell viability tests of nanoparticle-treated cells. 3-(4, 5-dimethyl-2-thiazolyl)-2, 5-diphenyl-2H-tetrazolium bromide (MTT) assay of (a) A549 carcinoma cells, (b) A2058 melanoma cells, (c) amniotic fluid-derived stem cells (AFSCs) and (d) human mesenchymal stem cells (hMSCs) after 24 and 48 hours of nanoparticle treatment (n≧4 in each group).

FIG. 7: Total retention of different-sized nanoparticles in the six vital organs and blood. As quantified using HPLC in (a) normal mice and (b) LPS-treated mice.

FIG. 8: Nanoparticle retention in peripheral tissues including (a) skin, (b) muscle and (c) fat and (d) urine quantified by HPLC. *, p<0.05; **, p<0.01; ***, p<0.001.

FIG. 9: The comparison of the retention of different sized nanoparticles in different organs following LPS treatment. An in vivo imaging system demonstrated the retention of nanoparticles of various sizes in different organs following normal saline, nanoparticles, and nanoparticle+LPS treatments.

FIG. 10: Size effect of nanoparticle retention under normal conditions or inflammatory conditions induced by LPS in (a) brain, (b) lung, (c) liver and (d) spleen. Retention was quantified using HPLC. *, p<0.05; **, p<0.01; ***, p<0.001.

FIG. 11: The tissue retention of nanoparticles of different sizes in the heart. Immunofluorescent staining of heart sections from normal or LPS-treated mice injected with normal saline or 20, 50, 100, 200, or 500 nm nanoparticles. Red: nanoparticles; green: isoleetin; blue: DAPI. Scale bar: 50 μm.

FIG. 12: The tissue retention of nanoparticles of different sizes in the kidney. Immunofluorescent staining of kidney sections from normal or LPS-treated mice injected with normal saline or 20, 50, 100, 200, or 500 nm nanoparticles. Red: nanoparticles; green: isolectin; blue: DAPI. Scale bar: 50 μn.

FIG. 13: The tissue retention of nanoparticles of different sizes in the skin. Immunofluorescent staining of skin sections from normal or LPS-treated mice injected with normal saline or 20, 50, 100, 200, or 500 nm nanoparticles. Red: nanoparticles; green: isolectin; blue: DAPI. Scale bar: 100 μm.

FIG. 14: The tissue retention of nanoparticles of different sizes in the muscle. Immunofluorescent staining of muscle sections from normal or LPS-treated mice injected with normal saline or 20, 50, 100, 200, or 500 nm nanoparticles. Red: nanoparticles; green: isolectin; blue: DAPI. Scale bar: 50 μm.

FIG. 15: Hindlimb ischemia-reperfusion disease model. (a) Procedure for generating hindlimb the ischemia- reperfusion disease model. (b) Blood flow rate of the ischemic legs before and after reperfusion. ***, p<0.001 vs. control.

FIG. 16: Retention of different-sized nanoparticles in nonischemic and ischemic hindlimb following 6 hours of reperfusion. *, p<0.05 vs. control (nonischemic).

FIG. 17: Nanoparticle distributions in normal or ischemic hindlimb muscles. Left column panels: nonischemic muscles; right column panels: ischemic muscles subjected to reperfusion for 6 hours. Red: nanoparticles; green: isolectin; blue: DAPI. Scale bar 100 μm.

FIG. 18: Nanoparticle distributions in normal or ischemic muscles. Left column panels: nonischemic muscles; right column panels: ischemic muscles subjected to hindlimb ischemia three-day reperfusion. Red: nanoparticles; green: isolectin; blue: DAN. Scale bar 100 μm.

### DETAILED DESCRIPTION OF THE PREFERRED EMBODIMENTS

Different nanoparticle properties, such as shape and surface charge, have been investigated to understand how to enhance the efficacy of nanoparticles in biomedical applications. However, there has not been a comprehensive study characterizing the size-dependency of nanoparticle biodistribution under different pathophysiologic conditions. Our study with fluorescent polystyrene nanoparticles revealed a size-dependent biodistribution of the nanopartieles that had been intravenously injected into normal mice. Further investigation showed that systemic inflammation induced by lipopolysaccharide changed the retention of the nanoparticles and led to redistribution in vital organs. Interestingly, we also observed a time-dependent distribution profile of the nanoparticles in a localized inflammatory hindlimb ischemia model. This model was validated by intravenous injection of polylactic-co-glycolic acid) (PLGA) nanoparticles that circulated into the ischemic areas. These unprecedented results show the importance of considering size when designing nanoparticles for use in nanoscale therapeutics and diagnostics.

### Methods

### Cell Viability Assay

Cells (A549 cells, A2058 cells, AFSCs and hMSCs) were seeded on to 12-well culture plates at a density of 2.6×104/cm2 in 1 ml total medium per well and allowed to adhere. 100 ul of 3-(4, 5-dimethyl-2-thiazolyl)-2, 5-diphenyl-2H-tetrazolium bromide (MTT) reagent (5 mg/ml 1XPBS) were added into each well and incubated for 2 hours at 37° C. After medium removal, 0.6 ml of DMSO was used to lyse cells and dissolve the formazan. The supernatant was collected and distributed into a 96-well plate at 0.2 ml each well for analysis. The absorbance was measured using ELISA reader (SpectraMax 340PC384, Molecular devices, USA) at 570 nm.

### Animals

The National Cheng Kung University Animal Care and Use Committee and the National Laboratory Animal Center approved all animal research procedures. FVB and nude mice of either sex (6 to 8 weeks, weight 22±0.6 g) were purchased from the National Laboratory Animal Center.

Quantification and imaging of nanoparticle biodistribution analysis

Fluorescent carboxylated polystyrene latex bead nanoparticles with uniform diameters of 20, 50, 100, 200, and 500 nm (Invitrogen or Polyscience) were used to investigate the biodistribution and retention of nanoparticles after intravenous injection into mice. These nanoparticles were non-degradable, thus excluding resorption as a variable. Nanoparticles were quantified by high-performance liquid chromatography (HPLC, Jasco, Essex, UK). Fluorescence microscopy and an in vivo fluorescence imaging system (IVIS 200, Caliper Life Sciences, Massachusetts, USA) were used to observe the nanoparticle biodistribution in the tissues and organs.

To quantify the nanoparticle retention, normal, healthy, mice were anesthetized with Zoletil (50 mg/kg; Virbac, France) and Rompun (0.2 ml/kg; Bayer Healthcare, Germany), and injected with one of the five sizes of nanoparticles through the jugular vein (150 μl/mouse). Mice were returned to their cages and received a normal diet and water for 4 hours. Major organs and tissues, including the brain, heart, lungs, liver, spleen, kidneys, skin, fat and blood, and urine, were harvested. These harvested tissues, organs and urine were digested in 0.5 or 3 ml of 1 M potassium hydroxide

(KOH) solutions at 60° C. overnight, depending on the sample. The total volumes of the brain, heart, lung, liver, spleen, kidney, blood, skin and fat were digested in a 0.5 ml volume. Due to the size of the liver, 3 ml of a KOH solution was required for complete digestion. All of the samples were then mixed with 0.5 ml of o-xylene for fluorescein extraction by sonication for 1 min and placed into a 60° C. oven for 15 minutes. The samples were vortexed and incubated at 60° C. for 5 min; this step was repeated twice. For the urine sample, 0.5 ml of xylene was added directly without KOH digestion. The preparation of these samples then followed the procedures described previously. Finally, all of the samples were centrifuged for 30 minutes at 14,000 RPM, and the supernatants were analyzed by HPLC.

HPLC standards were measured by sampling 10, 40, 80, 160, and 200 μg of 20, 100, 200, and 500 nm nanoparticle solutions and 12.5, 25, 75, 100, and 150 μg of 50 nm nanoparticle solutions. The extraction procedures for nanoparticle standards were identical to the protocol described above. The relative amount of nanoparticle retention in each sample was calculated using the calibration standard curves.

### Systemic Inflammation and Hindlimb Ischemia-Reperfusion Injury Model

Mice were anesthetized by injecting Zoletil (50 mg/kg; Virbac, France) and Rompun (0.2 ml/kg; Bayer Healthcare, Germany) before surgery was performed. For the systemic inflammation model, lipopolysaccharide (LPS, 5 mg/kg; Sigma, USA) was injected into the mice through the tail vein, followed with intravenous injection of nanoparticles after 24 hours. After four hours, mouse tissues and organs were harvested for sample preparation, as described above.

The hindlimb ischemia-reperfusion model was produced by ligating the right femoral artery of the unilateral right leg for 1 hour using a surgical suture. The sutures were then released for reperfusion for 6 hours, 1 day, or 3 days. Hindlimb blood flows were measured by laser Doppler (O2C flow meter, LEA Medizintechnik, Giessen, Germany) before and after surgery to confirm vessel occlusion. After 6 hours, 1 day and 3 days after reperfusion, the blood flow rates of both the injured leg (ischemic, right side) and the normal leg (nonischemic, left side) were measured, and different-sized nanoparticles were administered in the same procedure as outlined above. The muscles of both legs were harvested 4 hours after the nanoparticle injection. Samples were prepared and analyzed in the same procedure outlined above.

### Poly(Lactic-Co-Glycolic Acid) Nanoparticles for the Hindlimb Ischemia-Reperfusion Study

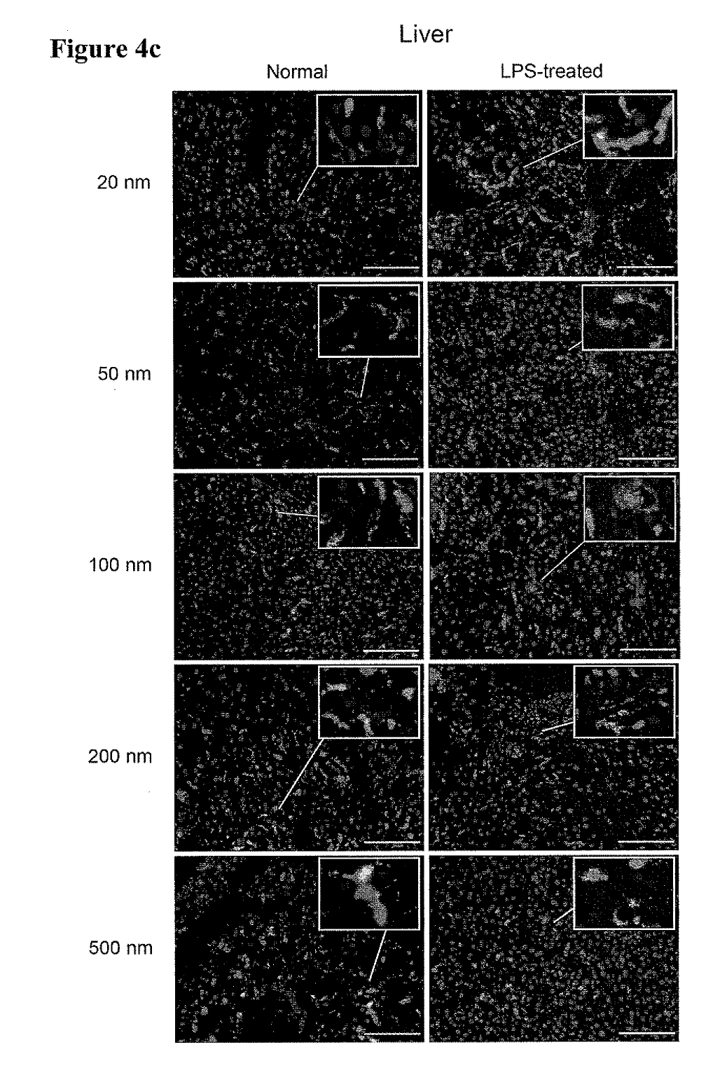

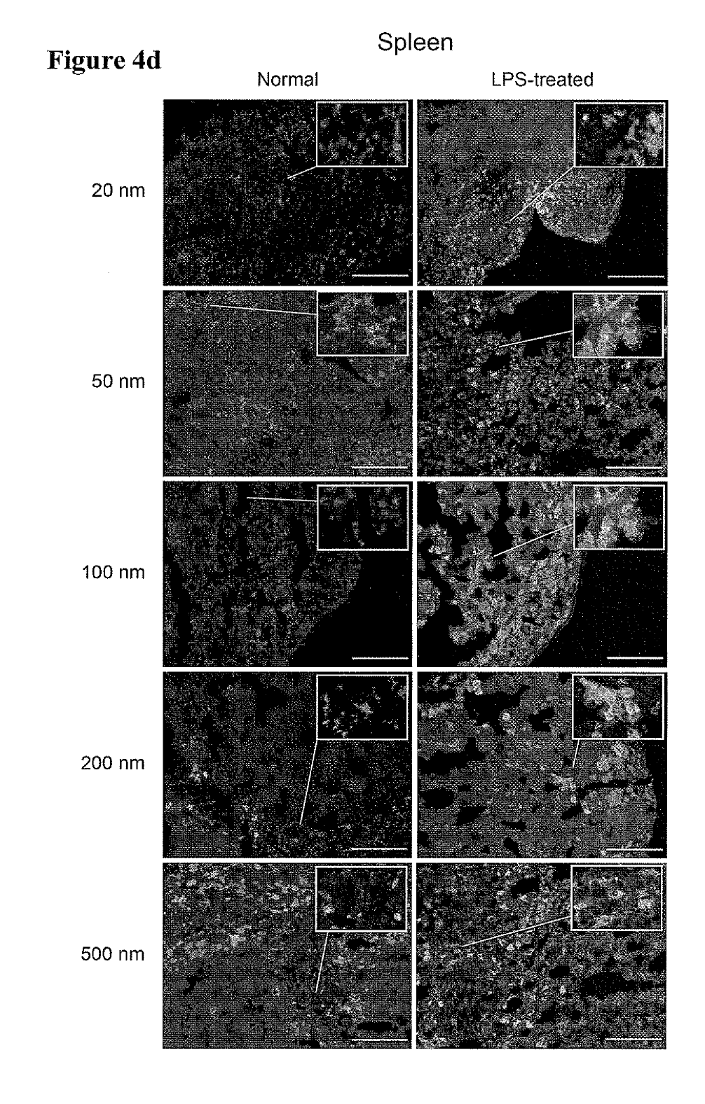

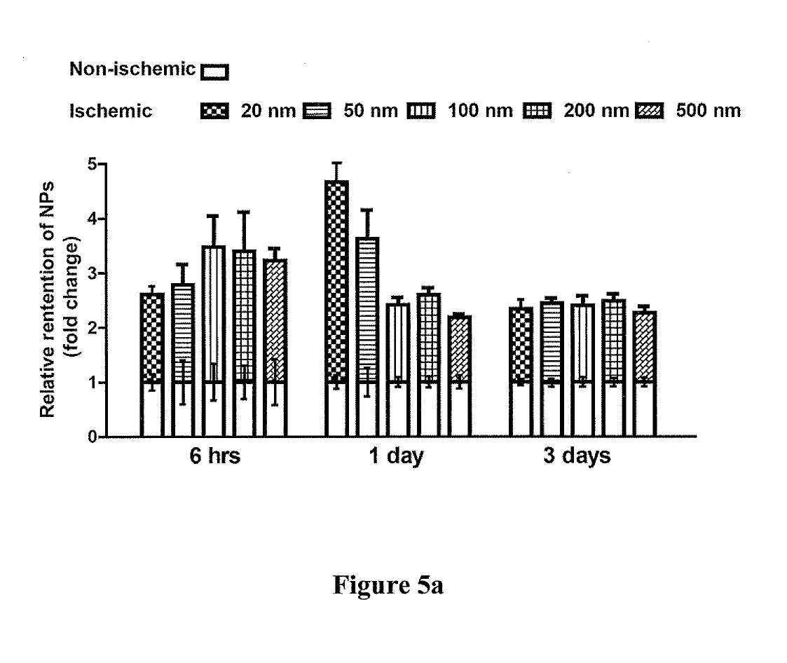

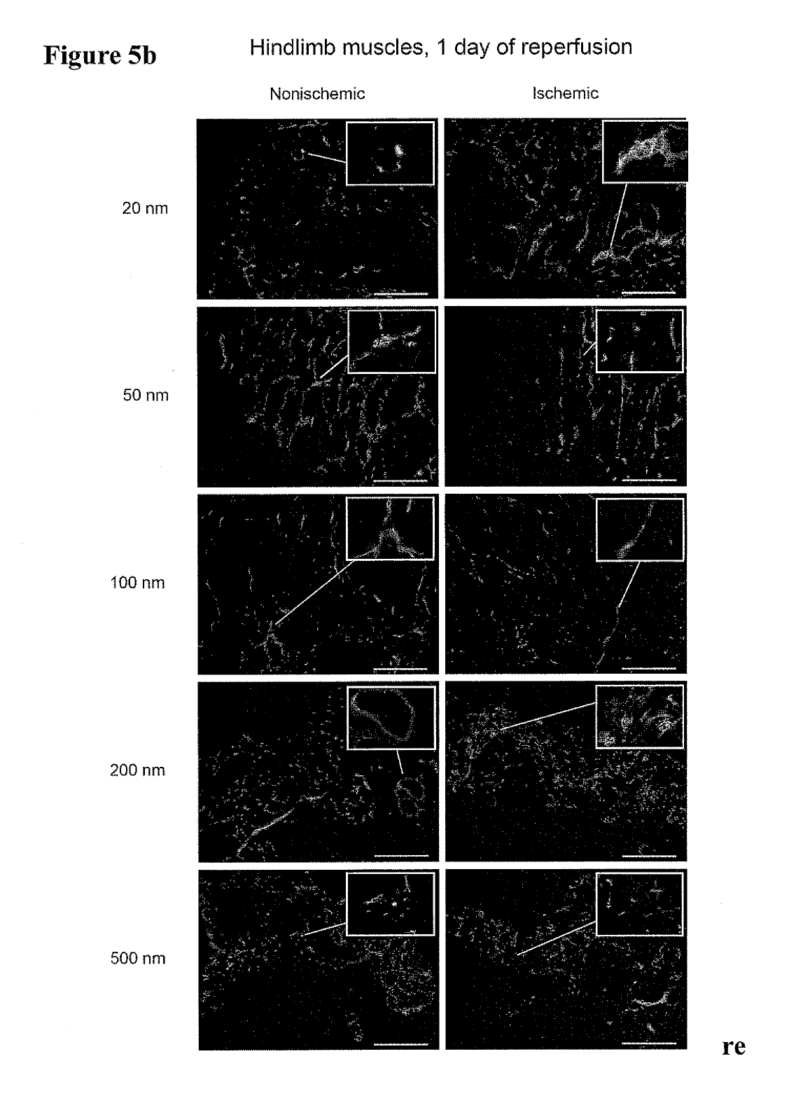

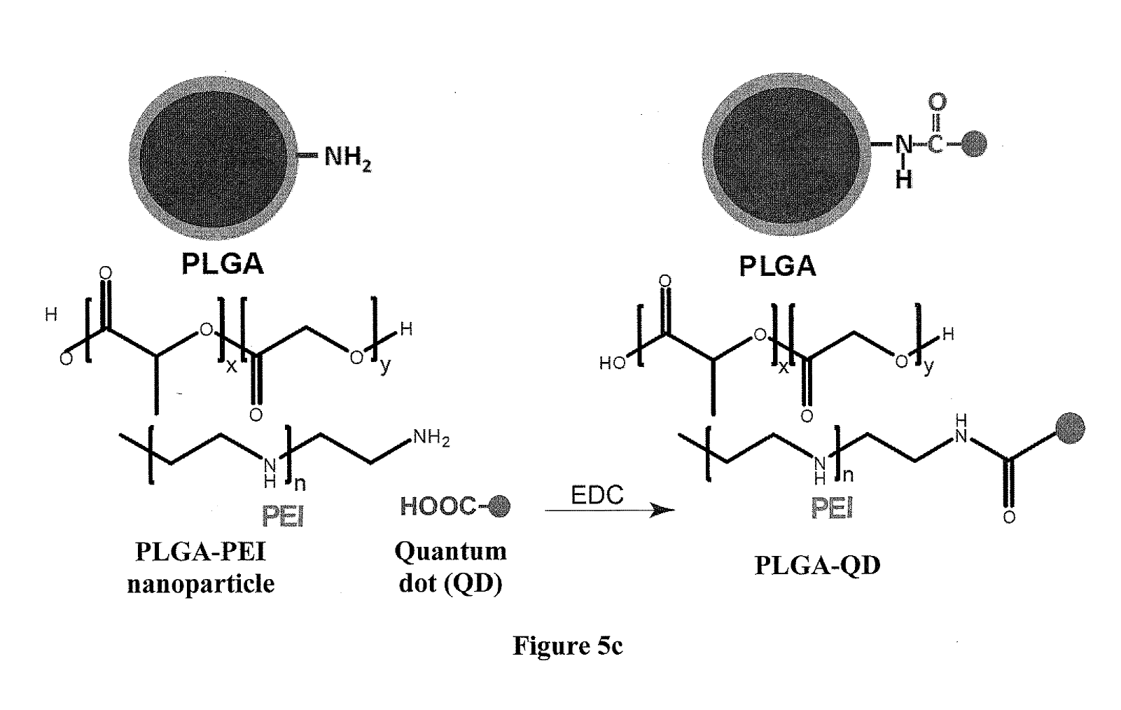

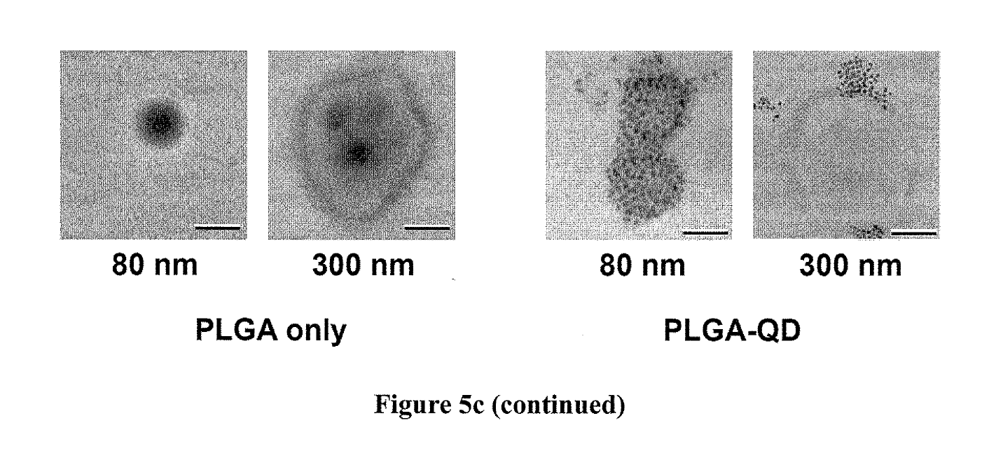

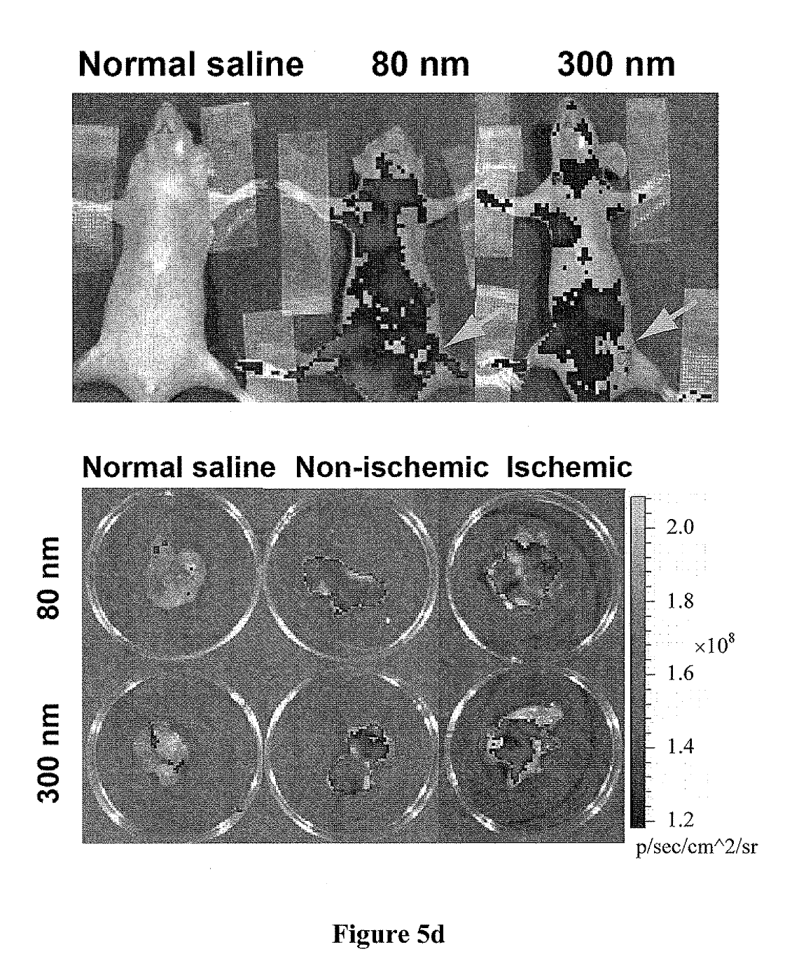

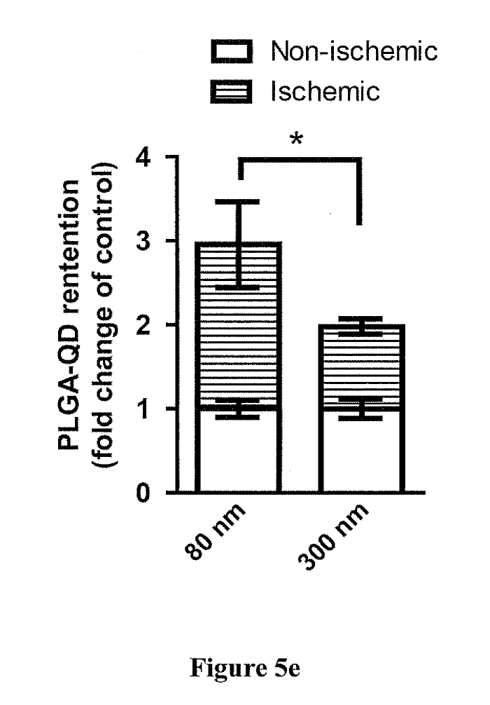

Poly(lactic-co-glycolic acid) (PLGA) was dissolved in 5 ml of acetone at a final concentration of 10 mg/ml. Ethanol/H₂O (50/50, % v/v) solution was added dropwise (1 ml/min) to the PLGA solution using a tubing pump and stirred at 400 RPM until turbid. After 5 minutes of additional stirring, the suspension was transferred into a glass beaker containing 20 ml of 1 mM polyethylenimine (PEI, Sigma) solution and homogenized at low speed for 20 minutes as previously described25. The solution was filtered through a 0.22 μm membrane. The produced nanoparticles were washed three times with deionized water. The functional group of QD-COOH was linked to the NH₂-terminated groups of PLGA NPs by adding 1-ethyl-3-(3-dimethylaminopropyl)-carbodiimide (EDC). The surface morphology of PLGA NPs and PLGA-QD NPs, as shown in FIG. 5c, was examined using transmission electron microscopy (TEM). Next, we obtained real-time images and tracked the model nanodrug carrier, PLGA-QD NPs, in the living animal. After injection of PLGA-QD NPs into the hindlimb of ischemia-reperfusion nude mice after a 1 day reperfusion, whole-body fluorescence images of the mice were analyzed. PLGA-QD NPs were excited at 605 nm and emitted at 660 nm.

### Statistical Analysis

Results are presented as the mean ±SEM. Statistical comparisons were performed with Student's t test. A probability value of p <0.05 was considered statistically significant. There were at least 6 animals in each group, unless specified.

### EXAMPLES

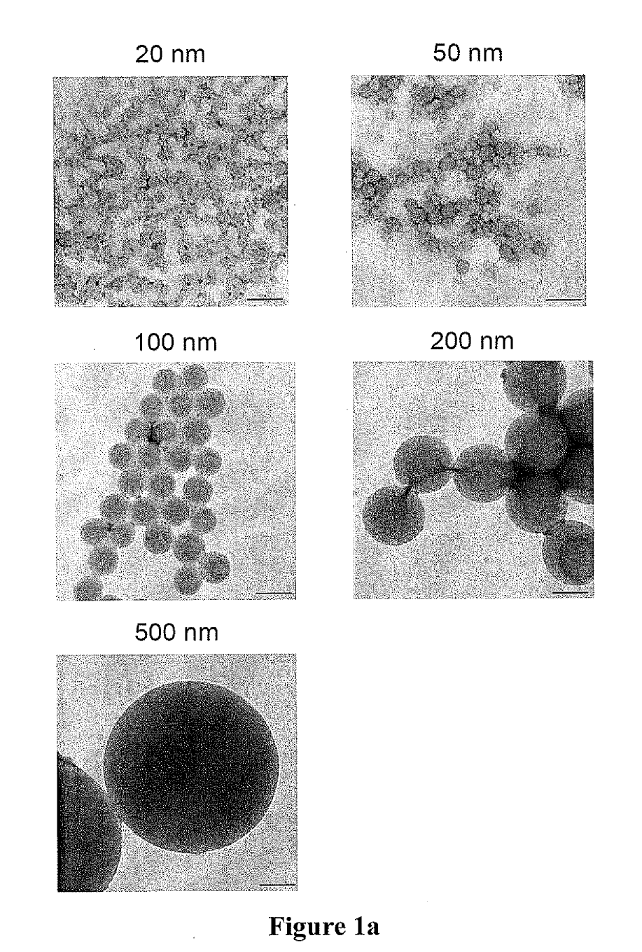

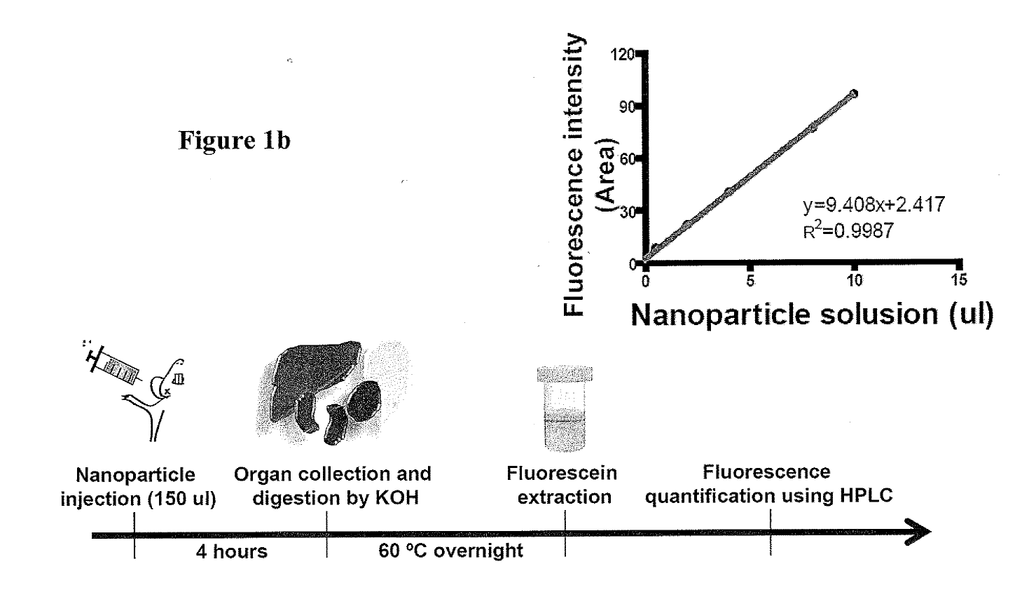

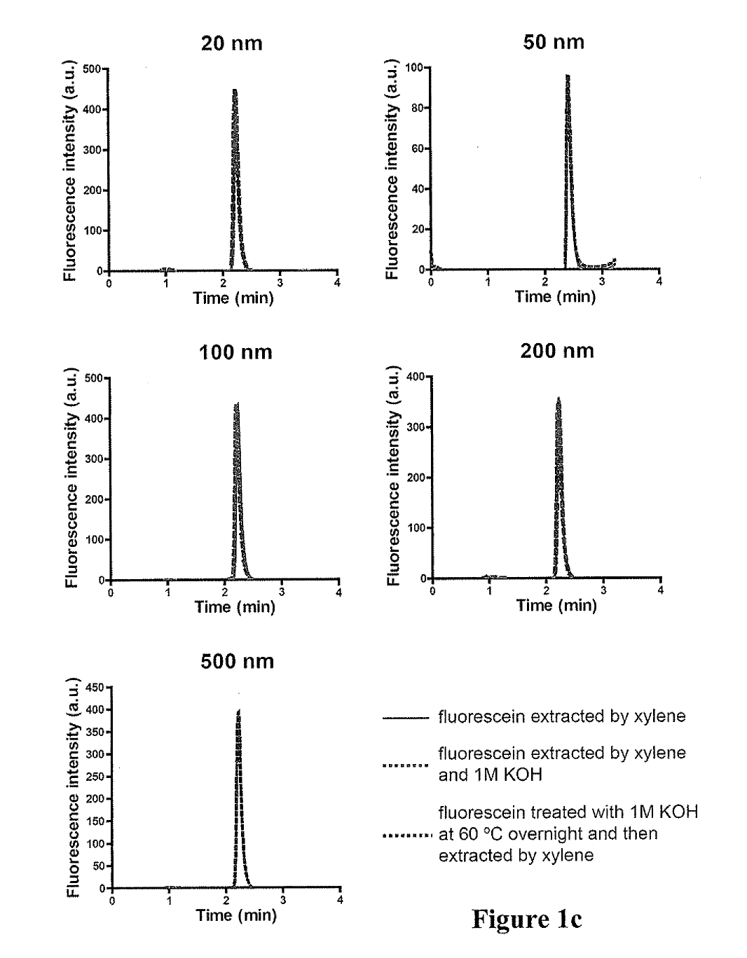

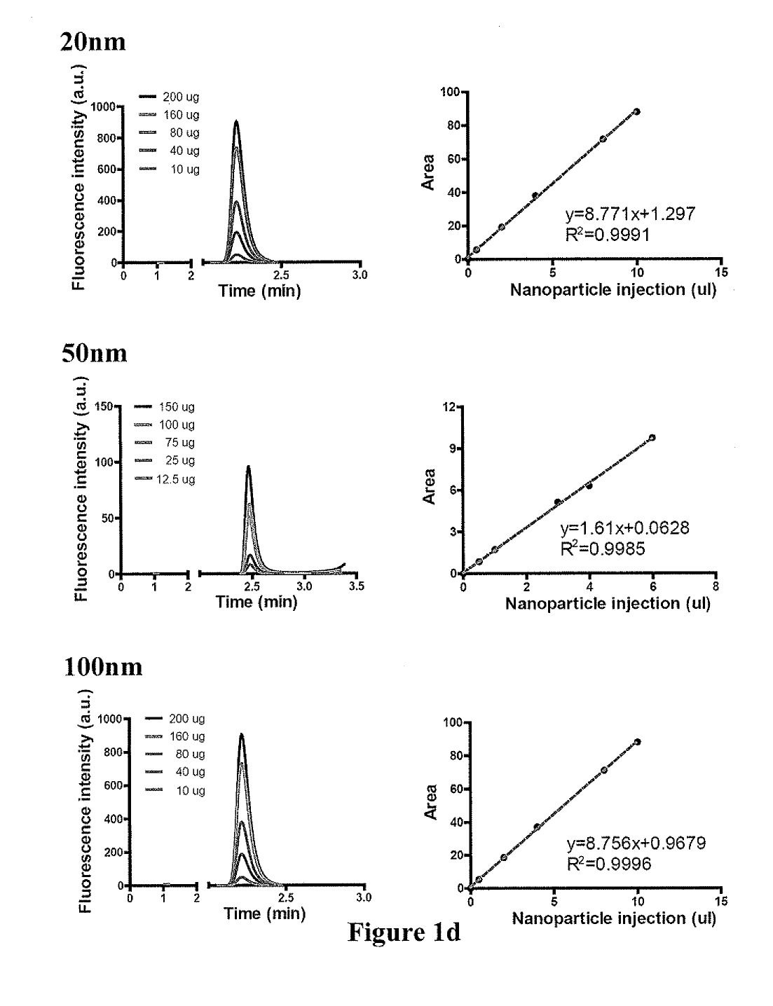

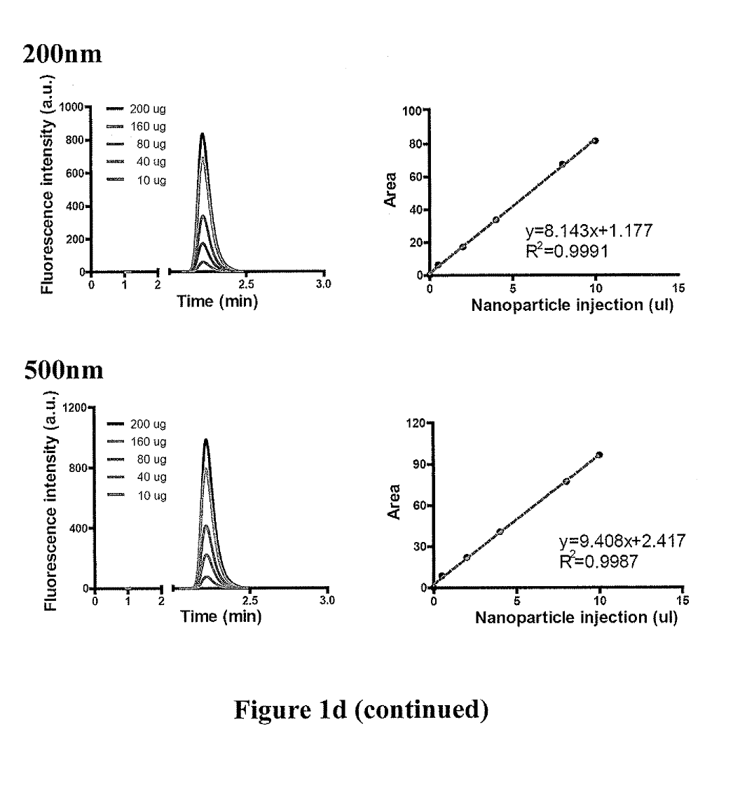

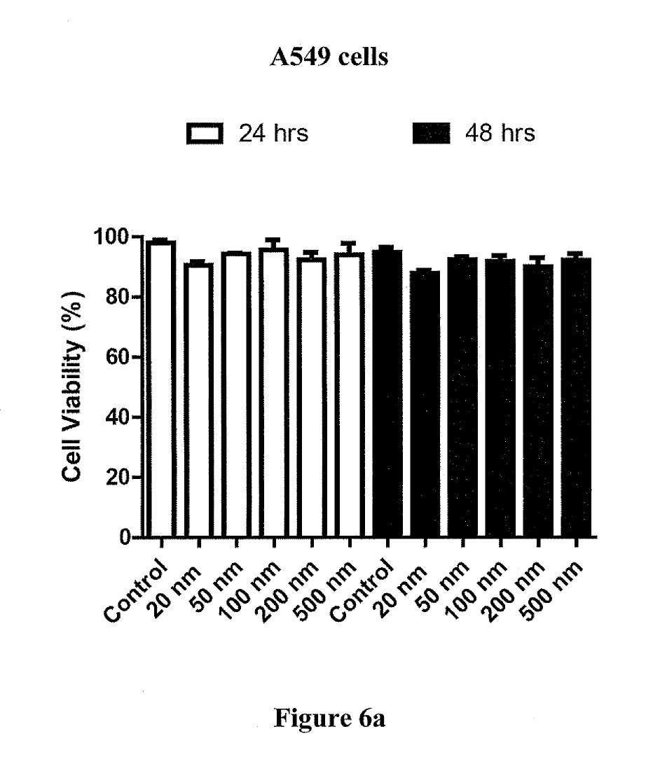

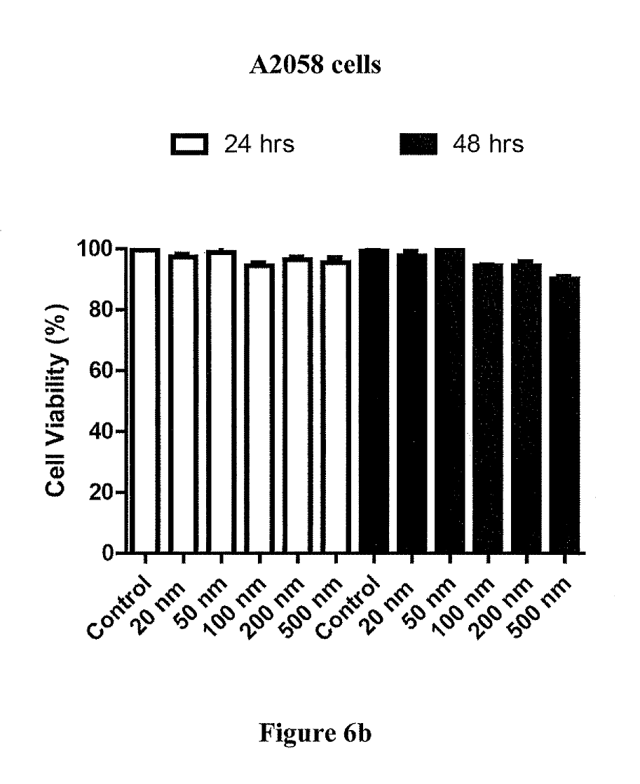

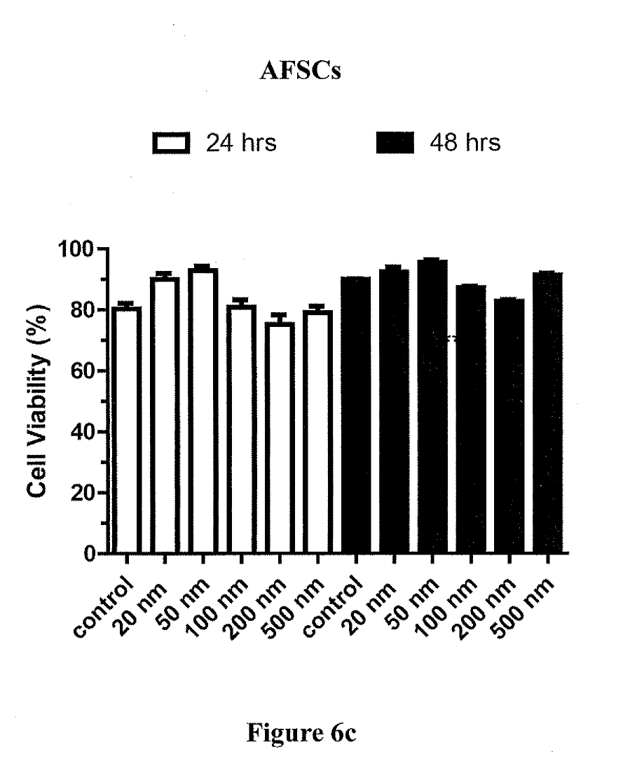

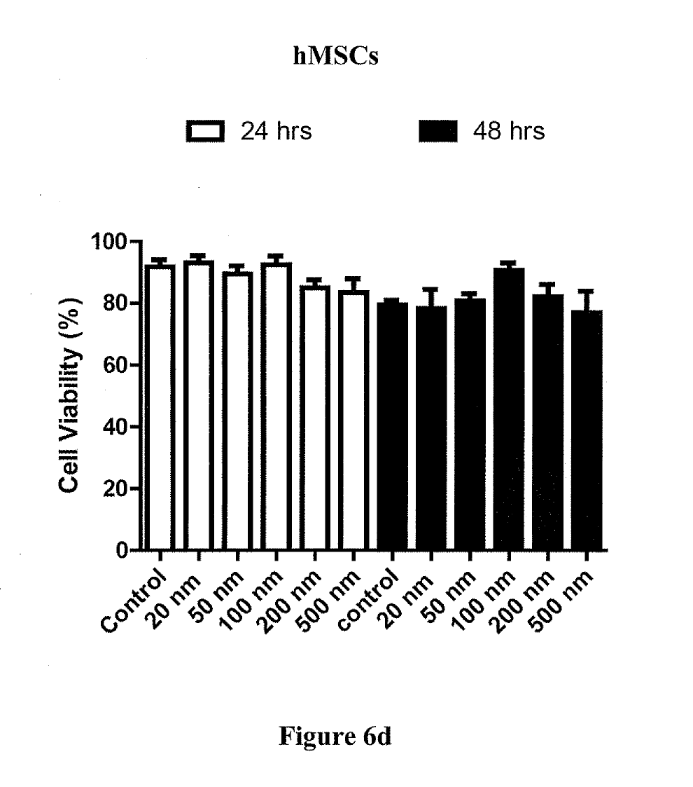

To characterize the size-dependent effects of nanoparticles, commercially available 20, 50, 100, 200 and 500 nm fluorescent polystyrene nanoparticles were acquired. The nanoparticle sizes and shapes were confirmed by transmission electron microscopy, which showed uniform size distribution and consistent spherical morphology (FIG. 1a). We detected minimal toxicity, which was similar for the nanoparticles of various sizes, using the (3-(4,5-dimethylthiazol-2-yl)-2,5-diphenyltetrazolium bromide) (MTT) assay for 4 different human cell lines, including A549 carcinoma cells, A2058 melanoma cells, cultured amniotic fluid-derived stem cells (AFSCs), and primary bone marrow mesenchymal stem cells (hMSCs) (FIG. 6). Therefore, we focused our studies on the in vivo characterization of different-sized fluorescent polystyrene nanoparticles following systemic injection.

As depicted in FIG. 1b, nanoparticles were injected into the jugular vein of mice to investigate the biodistribution of the nanoparticles. Organs and tissues were collected and digested in 1 M KOH overnight at 60° C. The samples were then mixed with o-xylene to dissolve the nanoparticles for fluorescein extraction after centrifugation. Supernatants were analyzed with high-performance liquid chromatography (HPLC) for nanoparticle quantification by measuring fluorescence intensity. To ensure that the protocol did not compromise the nanoparticle fluorescence signal, stock nanoparticles were treated with KOH and o-xylene at 60° C. overnight. HPLC analysis revealed an excellent alignment of the differentially treated nanoparticle samples, indicating that neither KOH nor o-xylene interfered with the fluorescence signal (FIG. 1c). Standard calibration curves were also established by extracting different amounts of a nanoparticle stock solution with o-xylene before HPLC analysis (FIG. 1d).

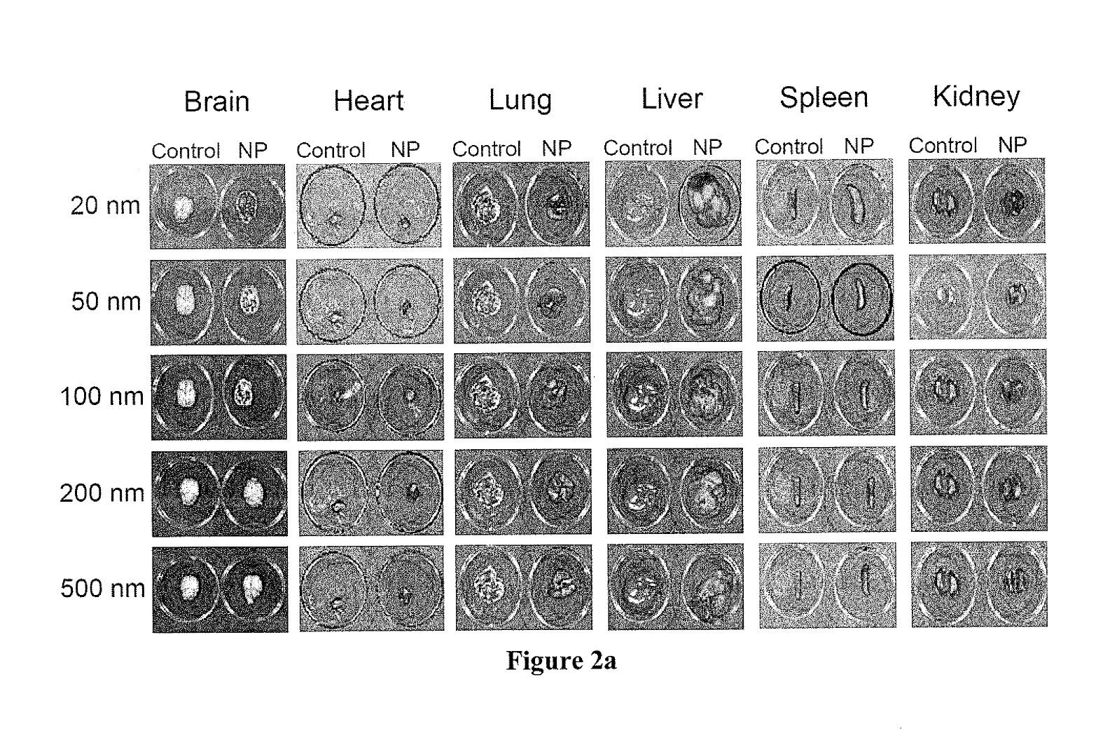

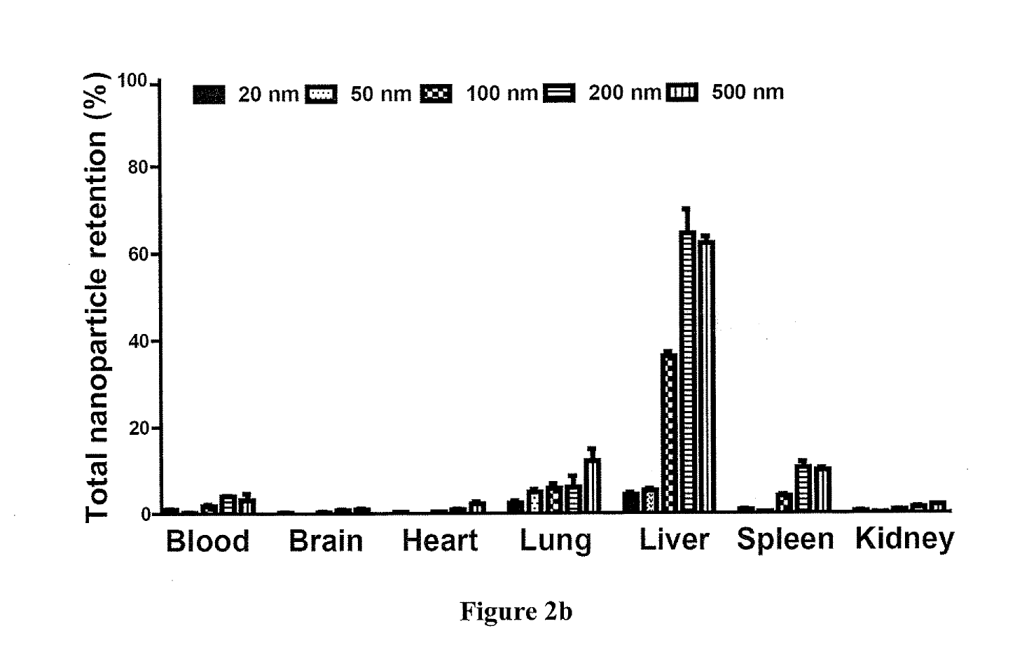

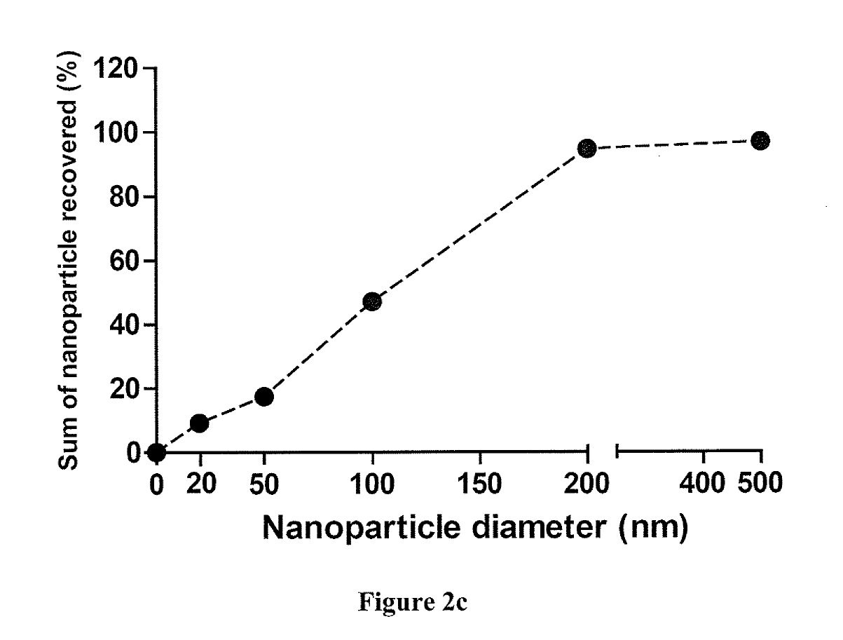

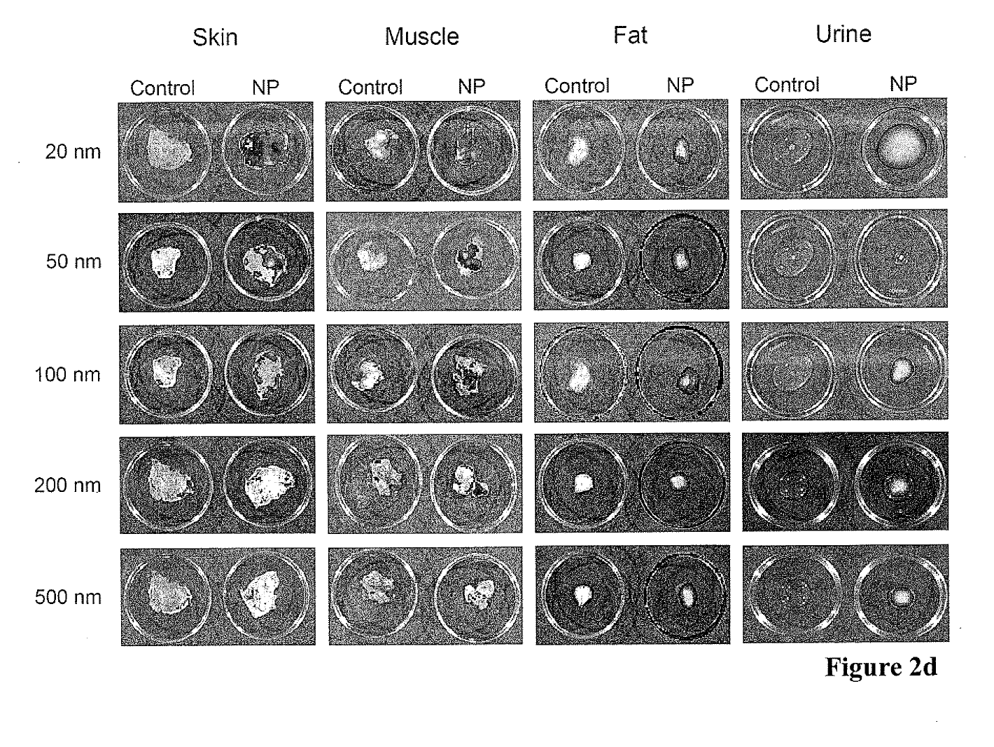

Nanoparticles are well-known for their short half-lives within the circulation and their rapid accumulation (within hours) in target tissues or organs⁹. To verify this biodistribution 3 mg (150 μl stock volume) of nanoparticles was injected into the jugular vein of healthy FVB mice and allowed to circulate for 4 hours. At this point, organs were collected for imaging with an in vivo imaging system (IVIS) or for sample preparation as outlined above for the HPLC analysis. IVIS images showed that the nanoparticles, regardless of size, were present in all of the vital organs, including the heart, lungs, liver, spleen, and kidneys (FIG. 2a). However, the fluorescence levels were extremely low in the brain when mice were treated with nanoparticles larger than 100 nm, suggesting that nanoparticles larger than 100 nm do not easily cross the blood-brain barrier (BBB). HPLC quantification confirmed the IVIS results, revealing that most of the nanoparticles were retained in the lungs, liver, and spleen in a size-dependent manner (FIG. 2b). A very steep cut-off size of 50 nm was determined for nanoparticle retention in the liver. When nanoparticle sizes changed from 50 nm to 200 nm, retention in the liver increased from approximately 5% to more than 60%. When designing nanoparticles for drug delivery, our results show that 200 nm is the optimal size for drug nanocarriers when targeting the liver. Anything larger is unlikely to increase retention in the liver.

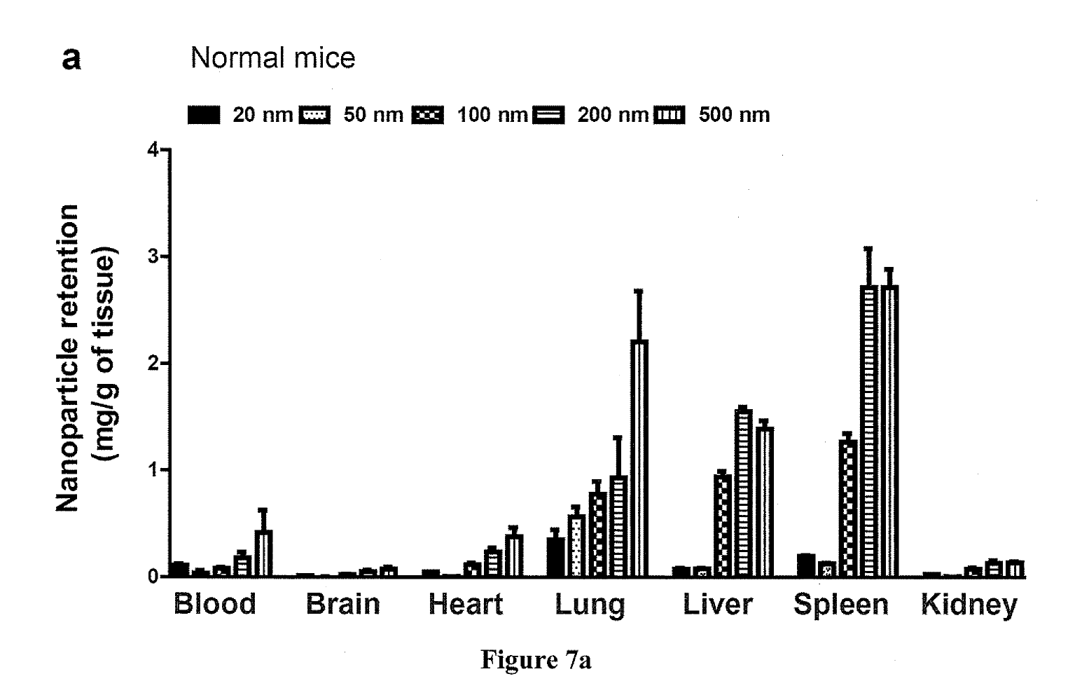

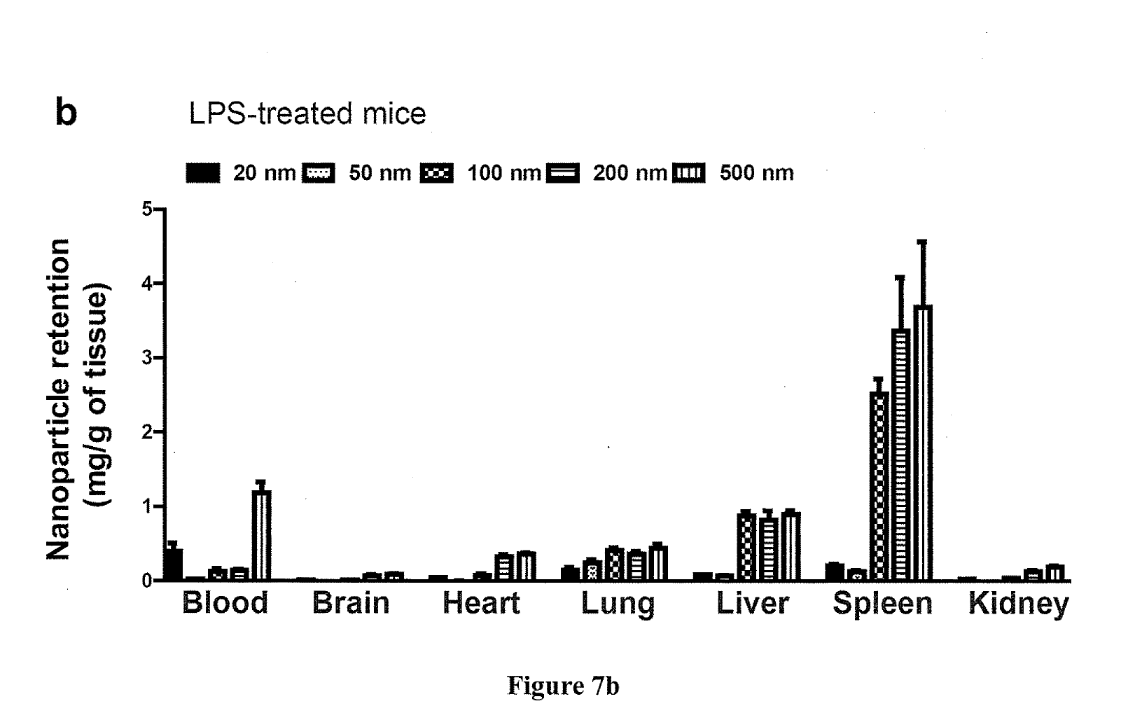

When the biodistribution of the nanoparticles was analyzed by a weight-to-weight ratio of nanoparticles to organs, nanoparticles were revealed to be more evenly distributed by nanoparticle density among the heart, lungs, liver, spleen, and kidneys (FIG. 7a). The heart and lungs retained the nanoparticles in a manner that was linearly proportional to the nanoparticle size, suggesting that the larger nanoparticles were blocked from exiting the capillaries to a greater extent. The spleen demonstrated a retention similar to the liver. Nanoparticle density increased dramatically from 0.1 mg/g to more than 2.7 mg/g when the nanoparticle size increased from 50 nm to 200 nm. Again, this result suggests that a 200 nm diameter is the optimum size when designing a nanoparticle to be retained by the spleen.

Detailed inspection of the HPLC quantification of brain nanoparticle retention revealed contradictory results. The IVIS images only showed the presence of nanoparticles smaller than 100 nm (FIG. 2a). However, HPLC results showed an increased retention in the brain proportional to the size of the nanoparticles (FIG. 2b). Therefore, we suspect that most of the nanoparticles greater than 100 nm were located in the center of the tissue sections that were sliced in the coronal plane. In contrast, nanoparticles smaller than 100 nm were retained primarily in the cerebral cortex and white matter. IVIS cannot image beyond a certain depth, depending on the tissue and the source of the fluorescence¹⁵. Therefore, in the present study IVIS was used only for preliminary biodistribution analysis, and. HPLC was used for precise biodistribution analysis and quantification. The IVIS images showed that nanoparticles accumulated in the center of the brain and were only preferentially distributed into the cortex when the size decreased.

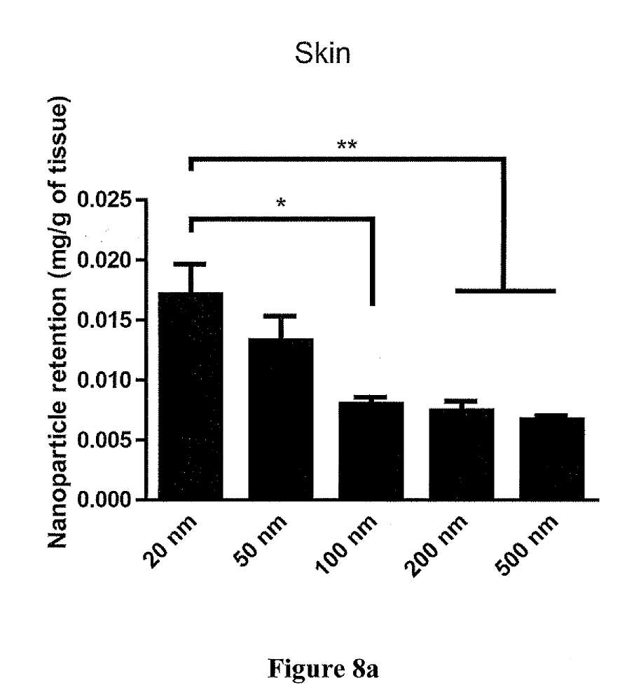

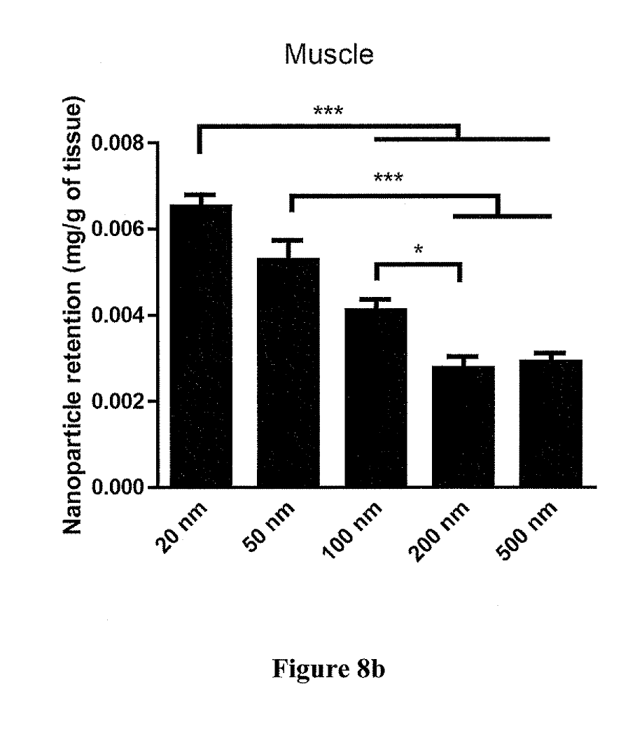

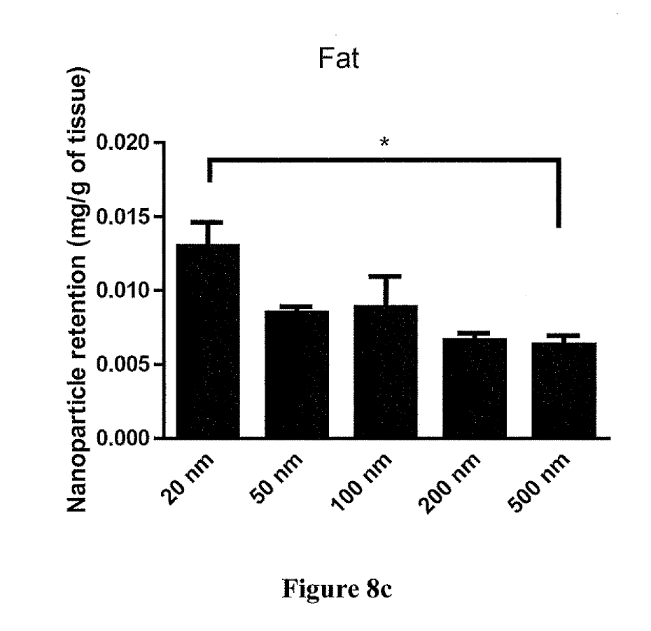

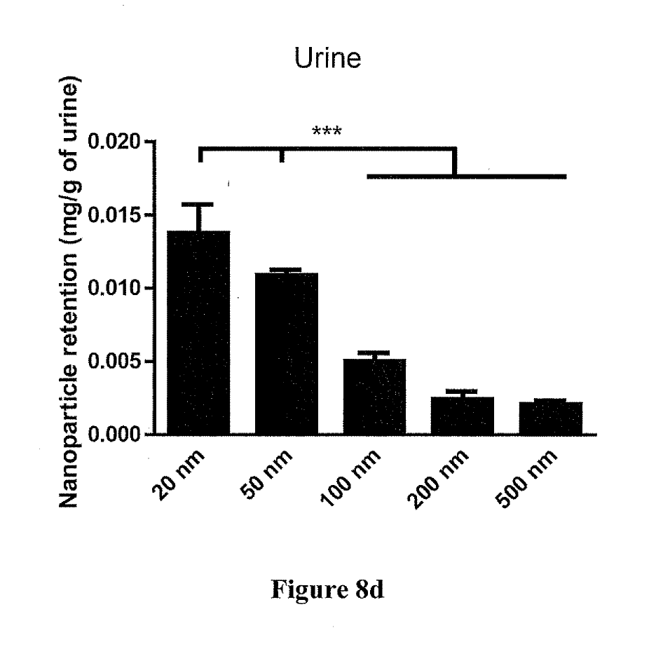

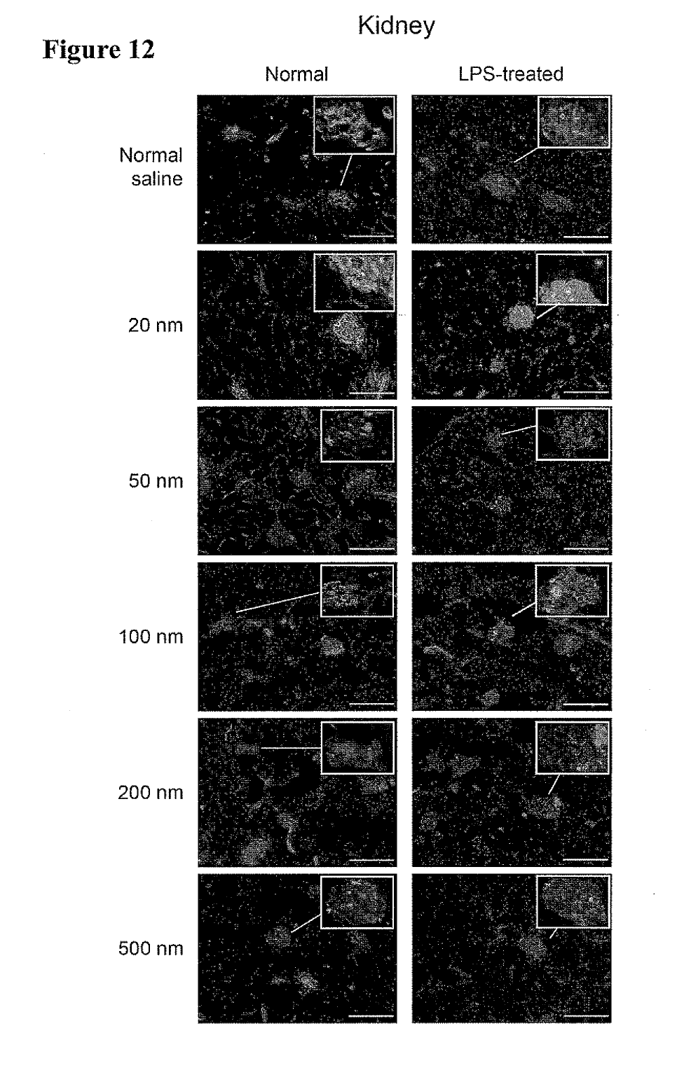

Most of the larger nanoparticles (100 nm or greater) were retained in the vital organs and the blood, and less than 20% of the smaller nanoparticles (below 100 nm) were recovered from our samples (FIG. 2c). To assess the fate of the remainder of the smaller nanoparticles, additional tissues and samples were analyzed, including skin, muscle, adipose tissue (fat), and urine. The IVIS images revealed a large amount of small nanoparticles present in the skin and muscle (FIG. 2d). HPLC quantification also indicated that the retention of nanoparticles in the peripheral tissues, including skin, muscle and fat, was inversely proportional to the size of the nanoparticles (FIG. 8a-d). Urine samples also contained more small nanoparticles than large nanoparticles, as one would expect. Nanoparticles distribute differently among organs and are excreted according to their size. Larger nanoparticles are more likely to be retained in the vital organs, either because of the size restriction of the renal system or of the organ itself. Smaller nanoparticles have the ability to permeate more easily throughout the vasculature, pass through the renal system into the urine, and distribute into the peripheral tissues. Components within the renal system, such as the glomerular endothelium or the glomerular basal membrane, filter small substances through a defined pore size¹⁶. As such, most nanoparticles within the kidneys were found to accumulate in the glomerulus (FIG. 12). Together with the finding that smaller nanoparticles were 2 to 3 times more likely to be found in the urine, the glomerulus was confirmed as a filter of 100 nm particles, which has been established by previous studies¹⁶.

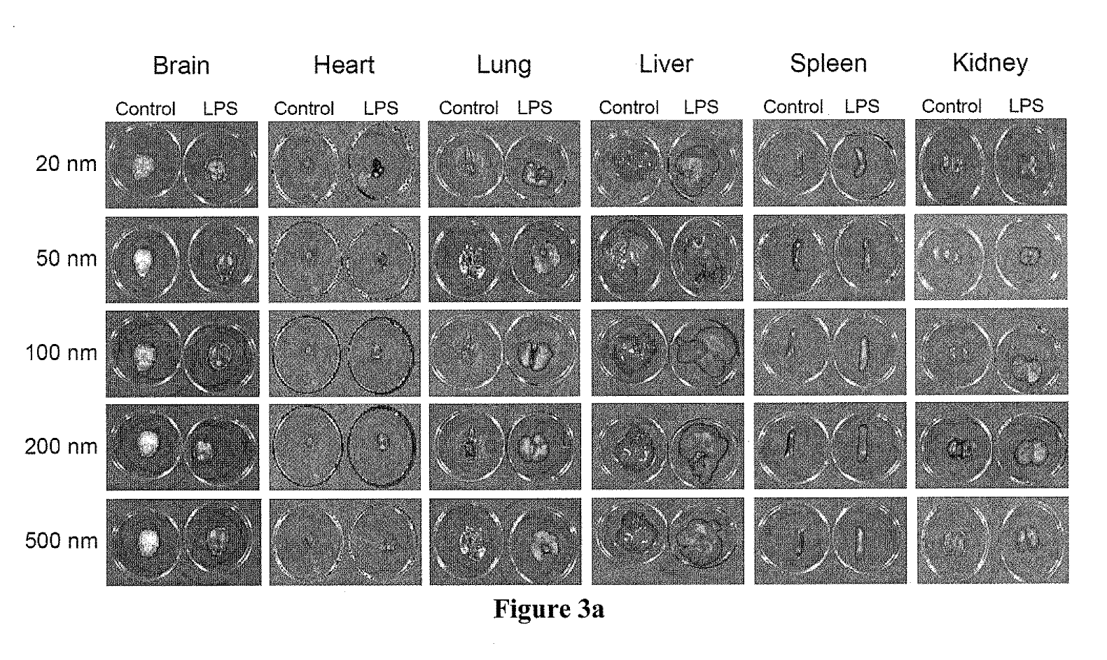

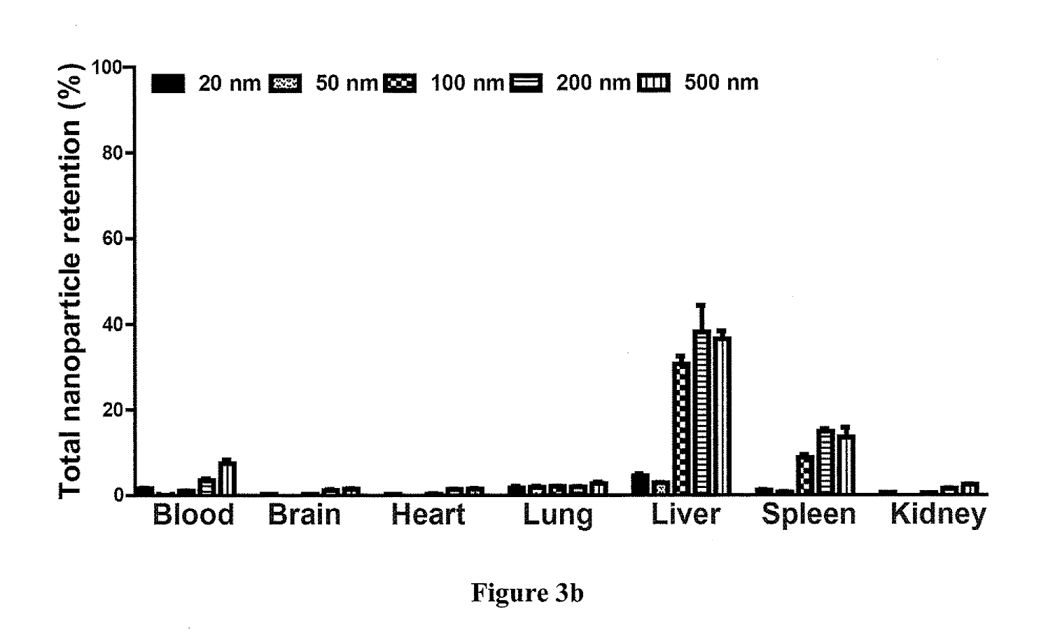

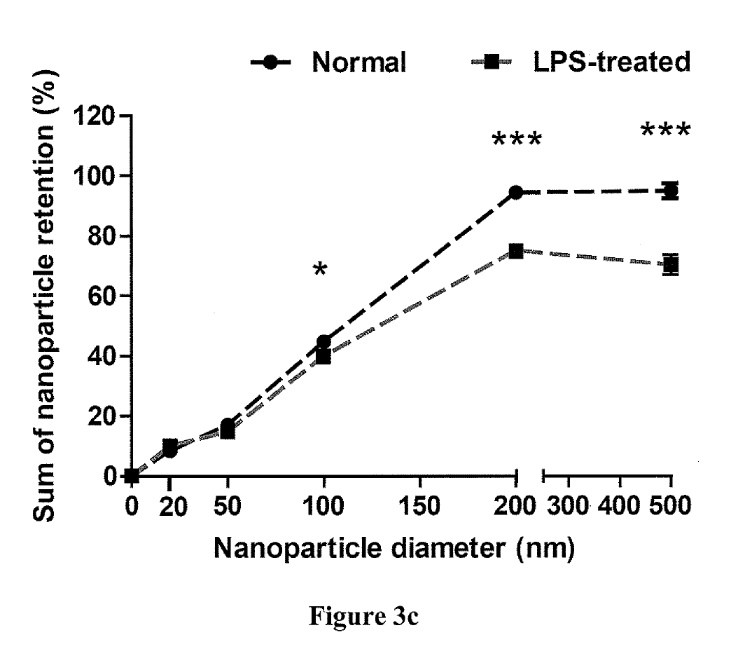

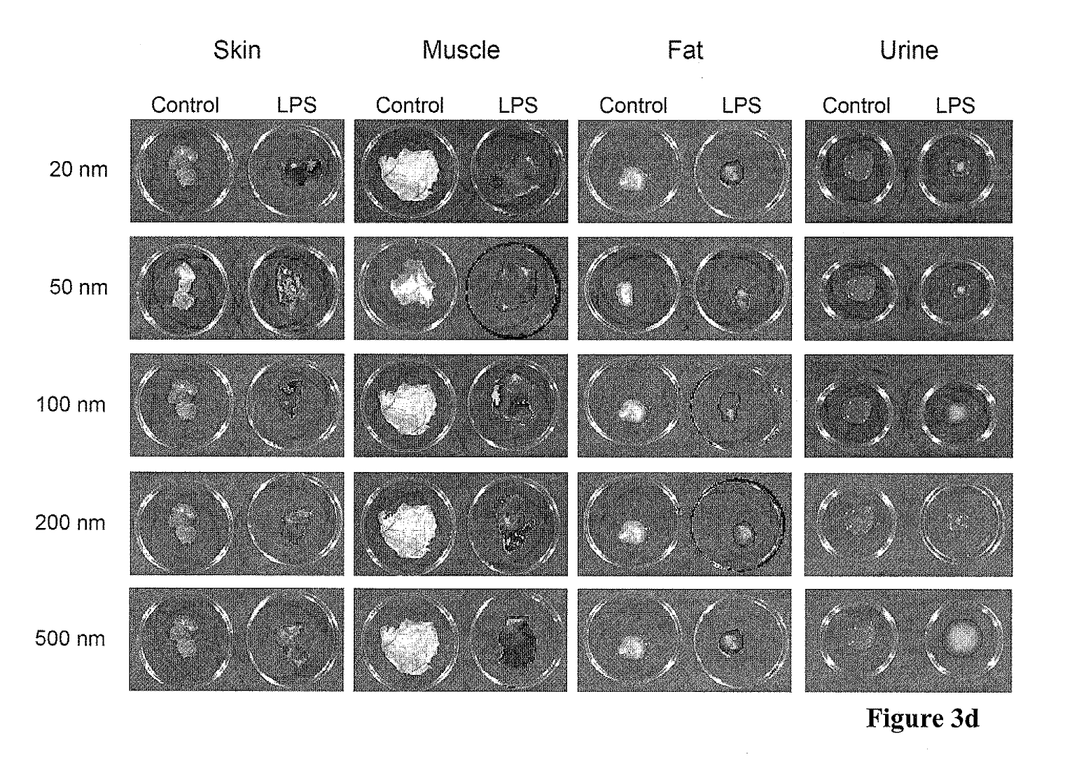

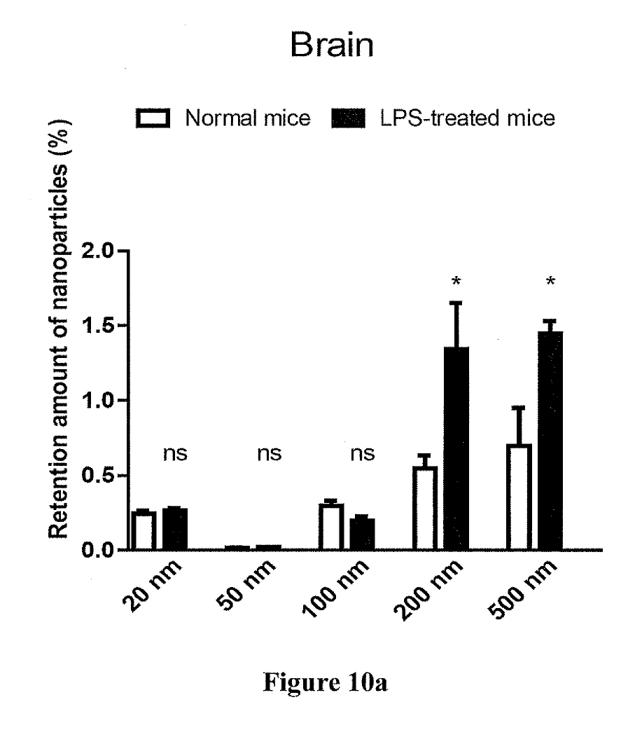

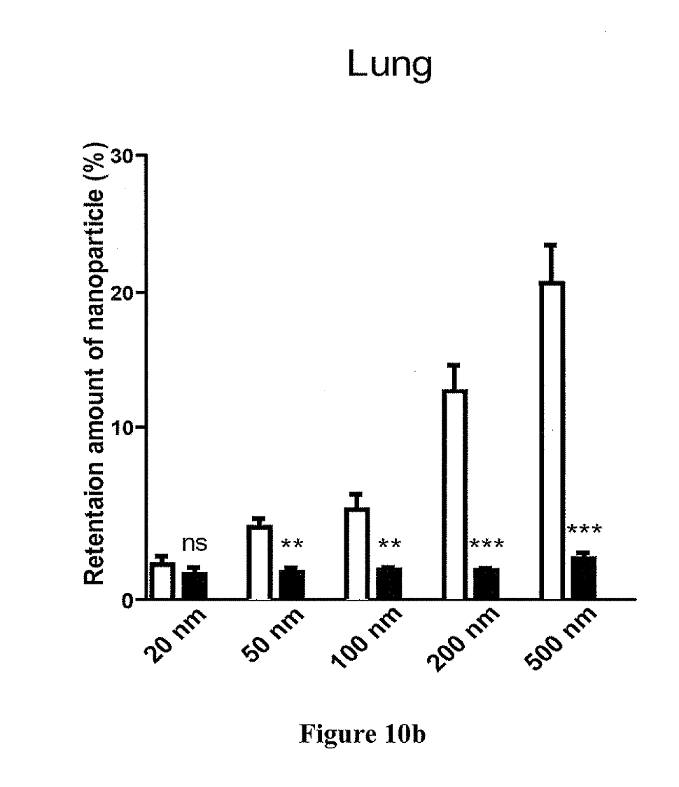

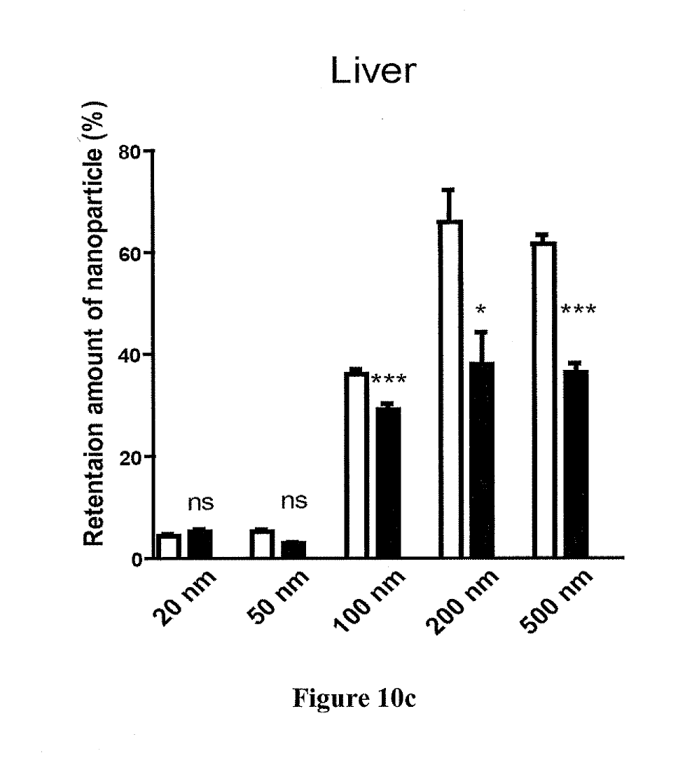

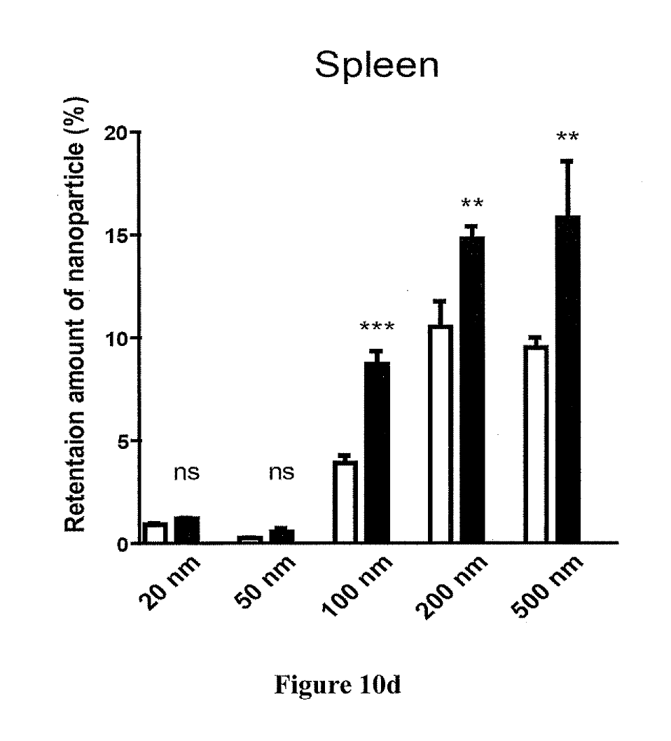

Drug nanocarriers have been designed to target tissues under specific disease conditions^{2,4,10,11}. A system under a disease condition responds differently to foreign bodies than a normal, healthy, system. The diseased system responds differently by altering microenvironmental conditions, varying cell behavior, and using signal transduction pathways that result in specific responses against the foreign bodies¹⁷⁻¹⁹. Thus, it is crucial that the uptake and distribution of nanoparticles be fully characterized in the diseased state of the model for the drug delivery system. To investigate whether a change in pathophysiologic conditions affects the biodistribution of nanoparticles, the same procedure was repeated as outlined above (FIG. 1b) for mice pretreated with the bacterial endotoxin lipopolysaccharide (LPS) to induce a systemic inflammatory response. IVIS imaging revealed a preliminary distribution profile of the nanoparticles that indicated the accumulation of the nanoparticles in all of the vital organs during systemic inflammation (FIG. 3a). Surprisingly, a distinctive signal was clearly detected for the 200 and 500 nm nanoparticle-injected brains after LPS treatment, and this signal was previously only slightly detectable. Furthermore, signals in the brain from all of the sizes of nanoparticles were more pronounced. HPLC analysis revealed that while the distribution of nanoparticles in healthy mice was size dependent, LPS-treated mice no longer exhibited the distinct size-dependent biodistribution (FIG. 3b and FIG. 7b). These results will be useful in drug delivery design for therapy targeted to the cerebral cortex. The diseased state of the body may allow more large nanoparticles to reach the cerebral cortex, but this effect is less pronounced for particles with a diameter of more than 200 nm. Beyond that size limit, the retention efficiency did not increase (FIG. 10a). In contrast, although smaller nanoparticles were capable of reaching the cerebral cortex, they were more likely to perfuse into the skin and muscles, similar to the case for normal tissues and organs. Additionally, small nanoparticles showed very high clearance rates and short half-lives (FIG. 2d, 3d and FIG. 8d).

In the heart, lungs, liver, spleen, and kidney, larger nanoparticles were evenly distributed (FIG. 3b and FIG. 7b). LPS-treated livers did not retain many nanoparticles compared to a normal liver. In fact, the spleen, which plays a vital role in the immune system, retained most of the nanoparticles when the mice experienced systemic inflammation. In the spleen, B cell proliferation is heavily induced by LPS treatment²⁰. A previous report also demonstrated the mechanisms of marginal zone antigen capture by B cells²¹. Besides B cells, dendritic cells may also be involved in antigen retrieval processes during early time points²². In our case, we suspect that the B cells may have facilitated nanoparticle transport through migration to follicular dendritic cells or through the retrieval capabilities of dendritic cells that migrated to the spleen.

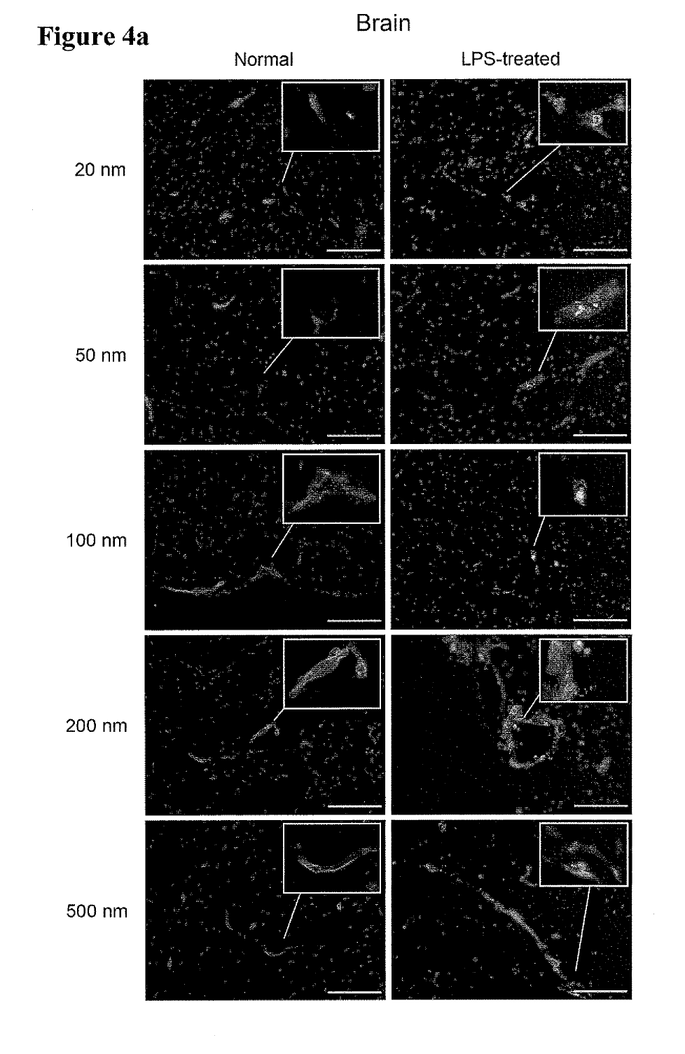

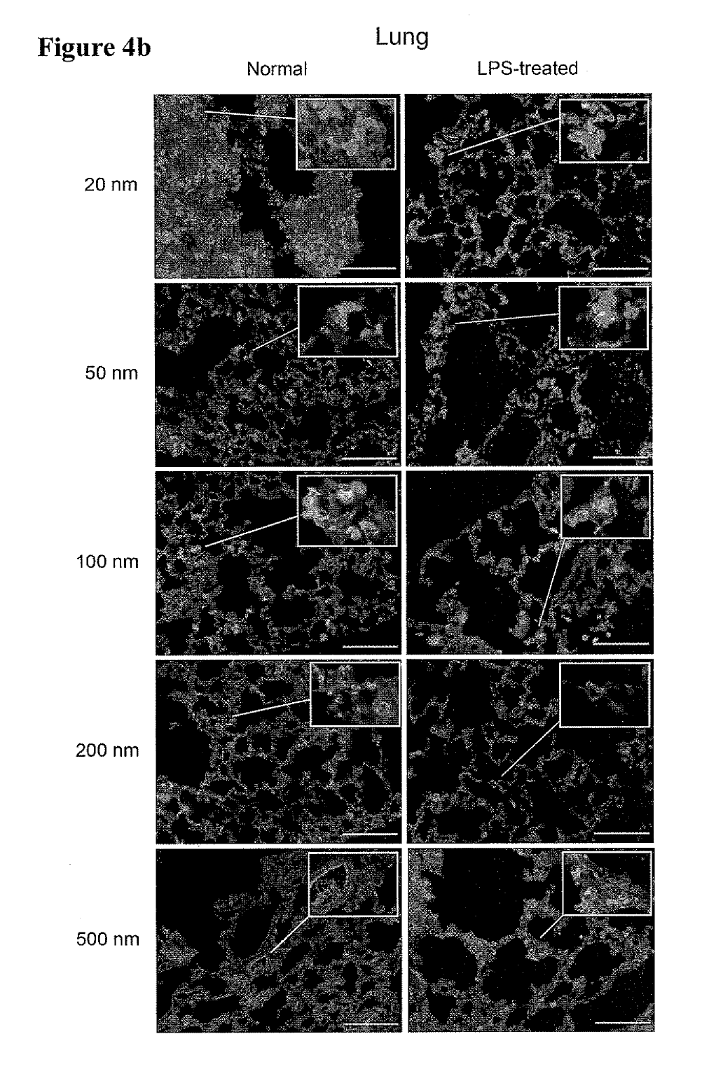

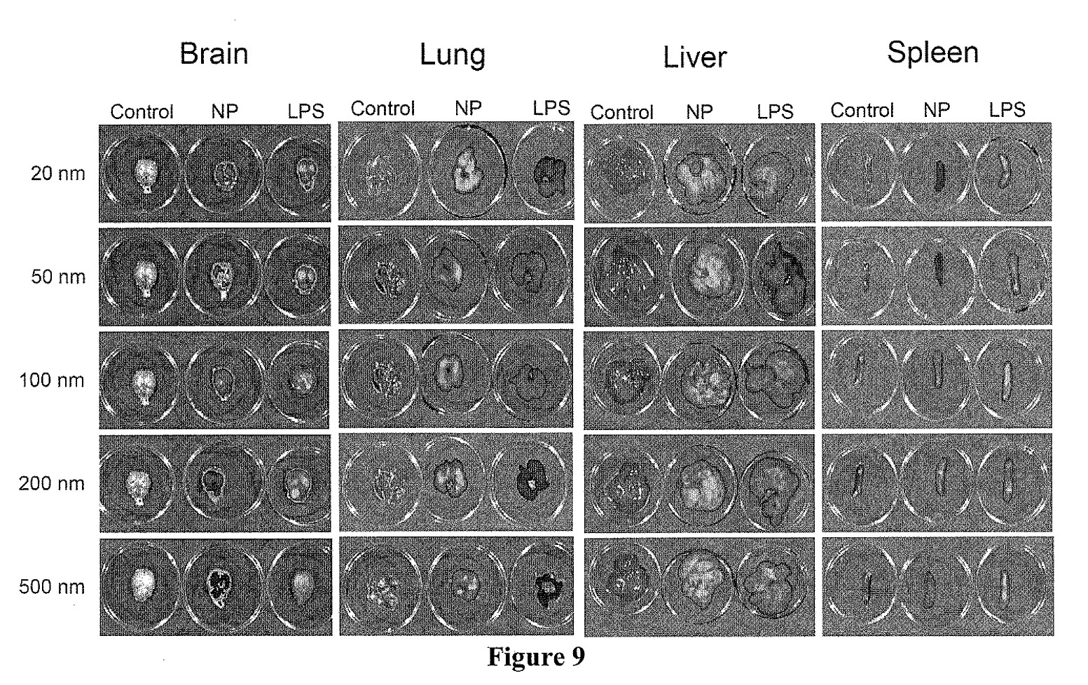

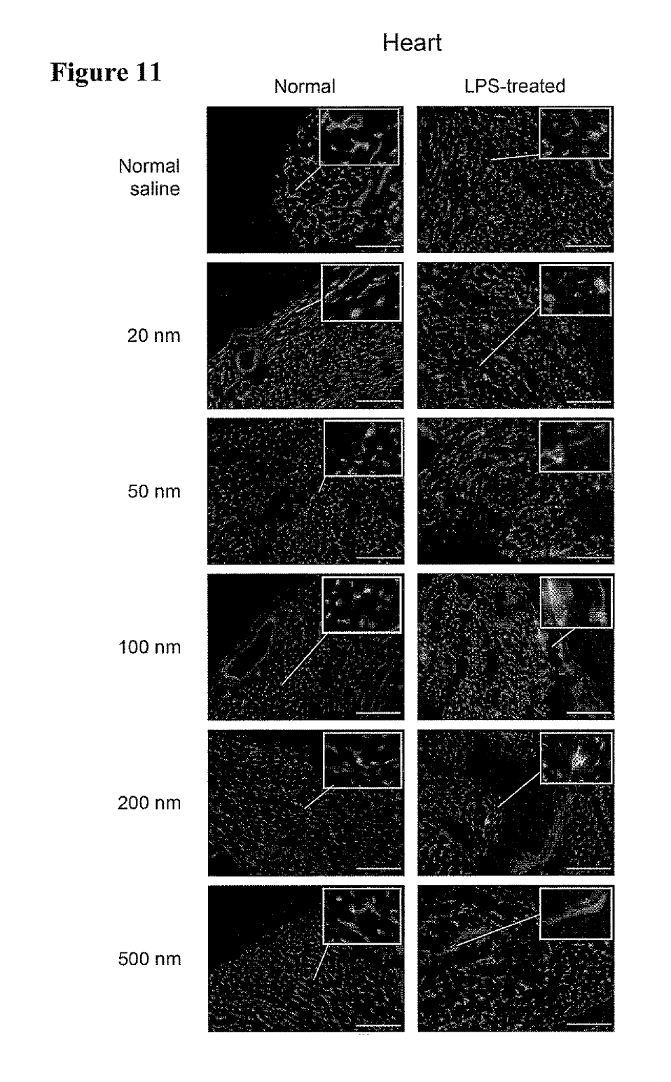

Tissue sections, IVIS imaging, and HPLC analysis also confirmed that more nanoparticles were retained in the brain and spleen but that fewer were retained in the lungs and liver of LPS-treated mice (FIG. 4a-d and FIGS. 9 and 10a-d). A summary of the nanoparticle retention in the vital organs revealed that in LPS-treated mice, there was a decrease in the nanoparticles in the vital organs compared to healthy mice (FIG. 3d). Interestingly, fewer nanoparticles accumulated in the heart during inflammation (FIG. 11). The increased blood flow and vasodilation, along with the high circulatory effect of the heart, may eject nanoparticles from this organ.

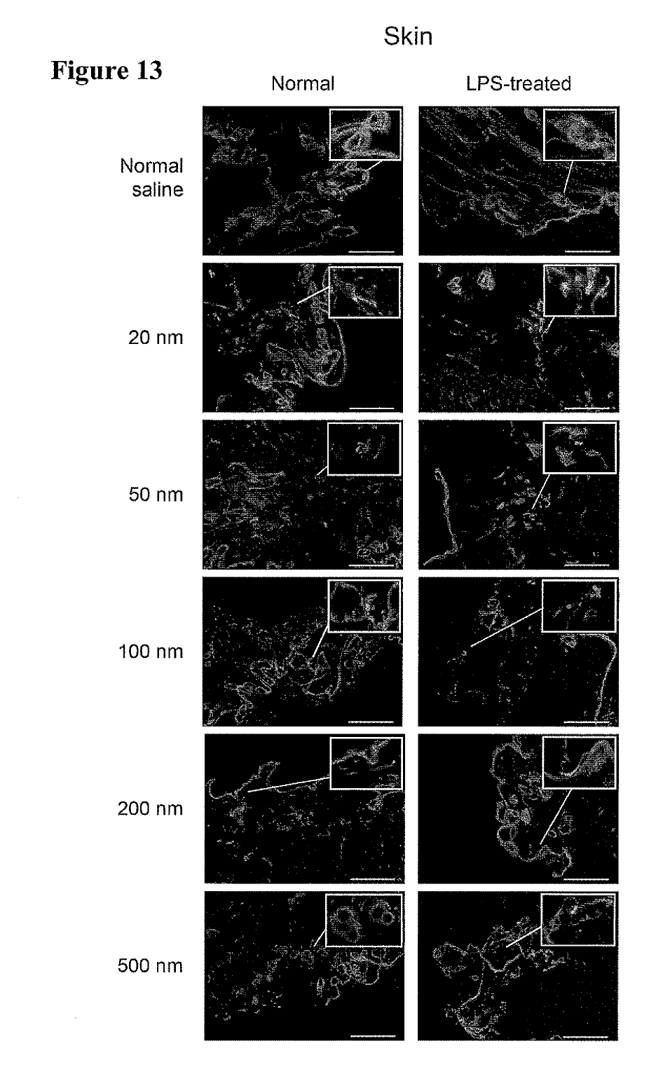

As mentioned above, additional tissues were analyzed. The IVIS images showed that larger nanoparticles were detected in the skin and muscle (FIG. 3e), which was confirmed by tissue sections (FIGS. 13 and 14). These larger nanoparticles were previously undetectable under the normal physiologic conditions in the mice. These results indicate that during a systemic inflammatory response, the mice experienced vasodilation and increased blood flow, which allowed larger nanoparticles to more readily permeate the peripheral tissues. The systemic inflammation induced by LPS changed the pathophysiologic conditions of the mice, causing a different fate for the nanoparticles.

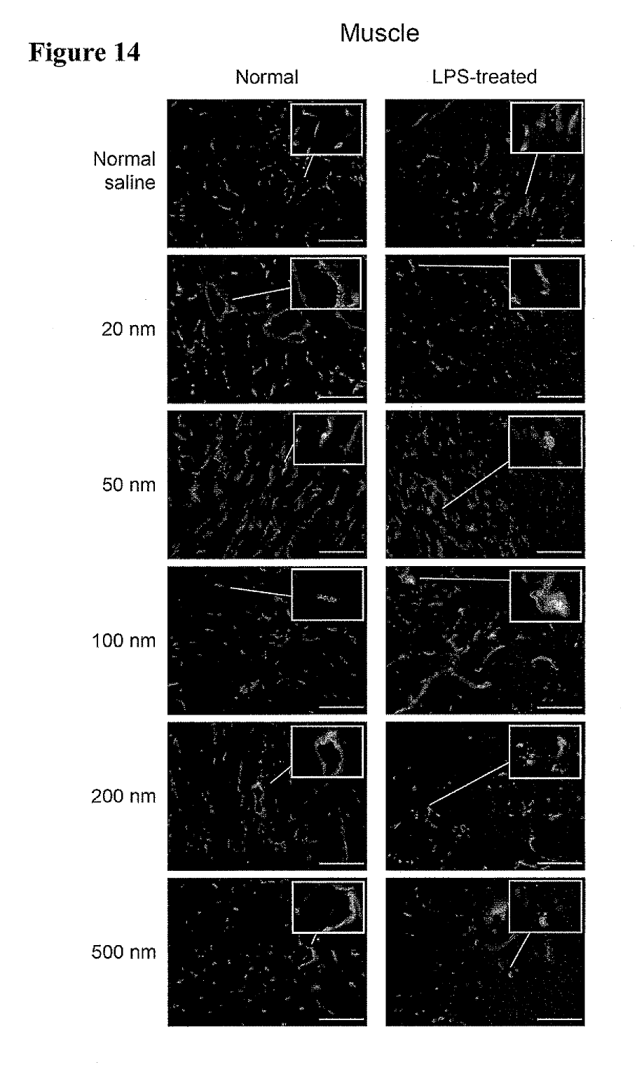

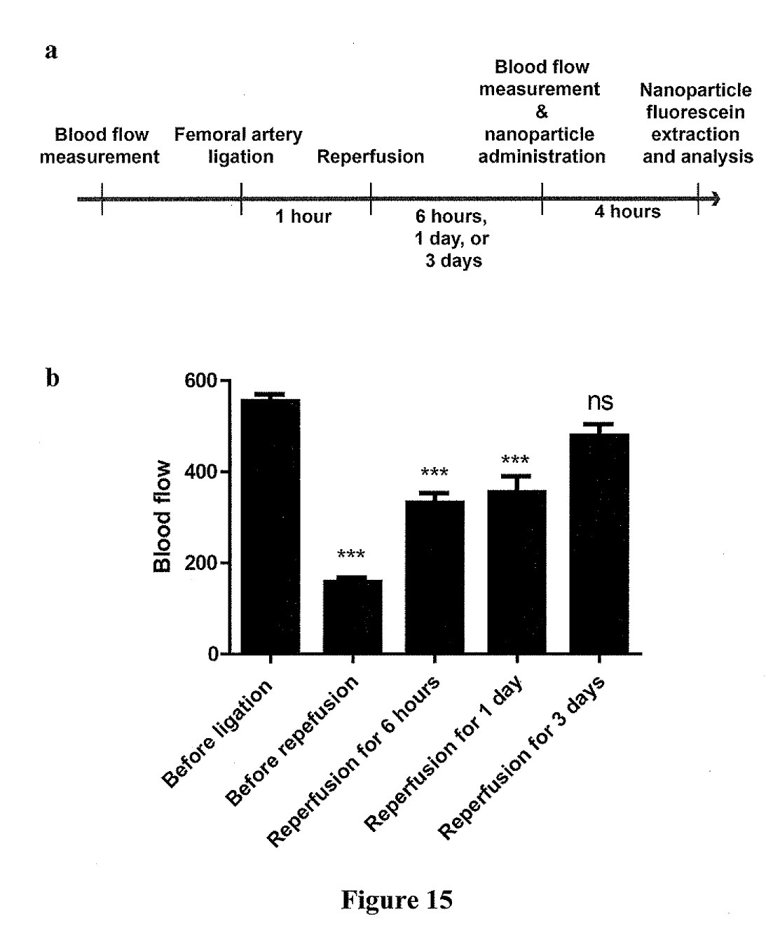

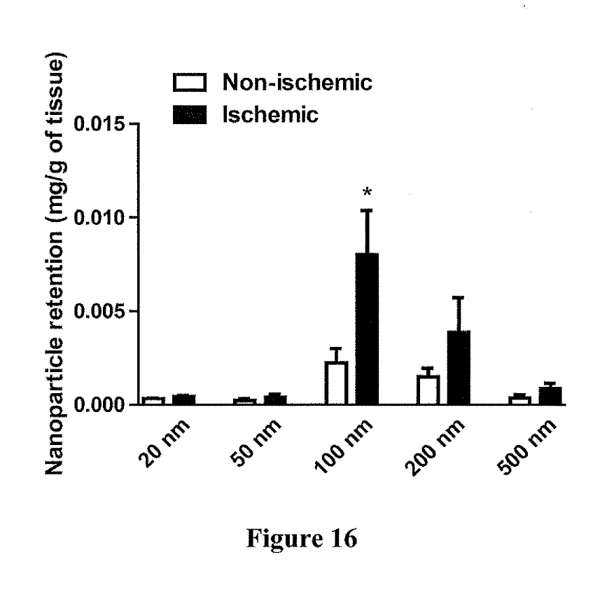

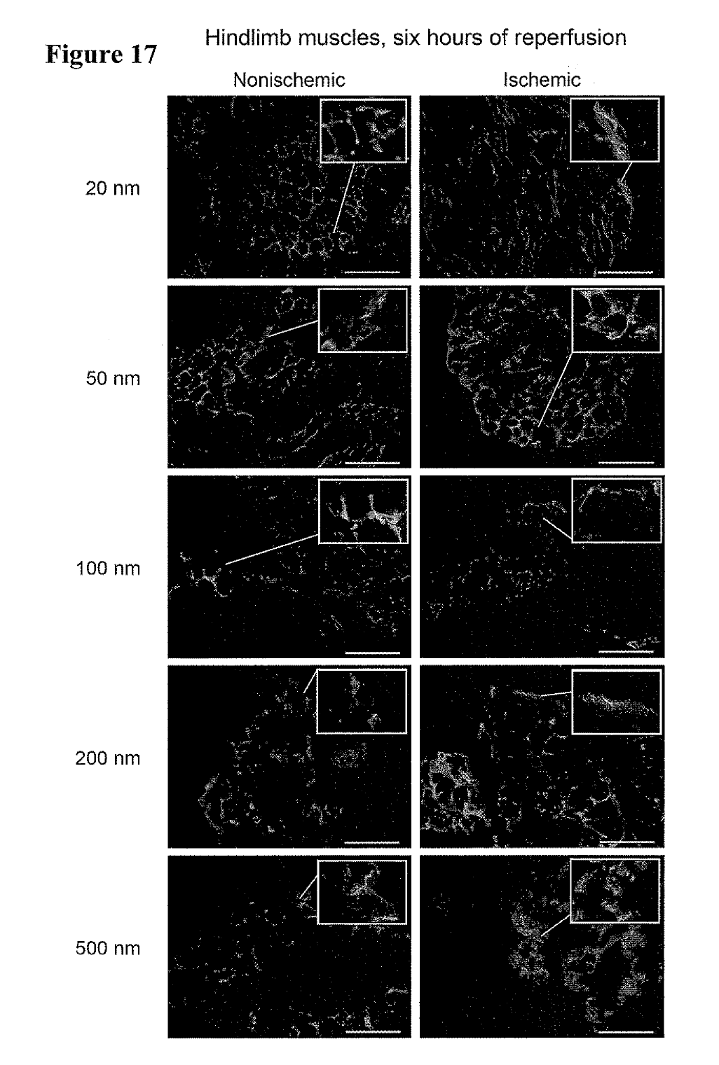

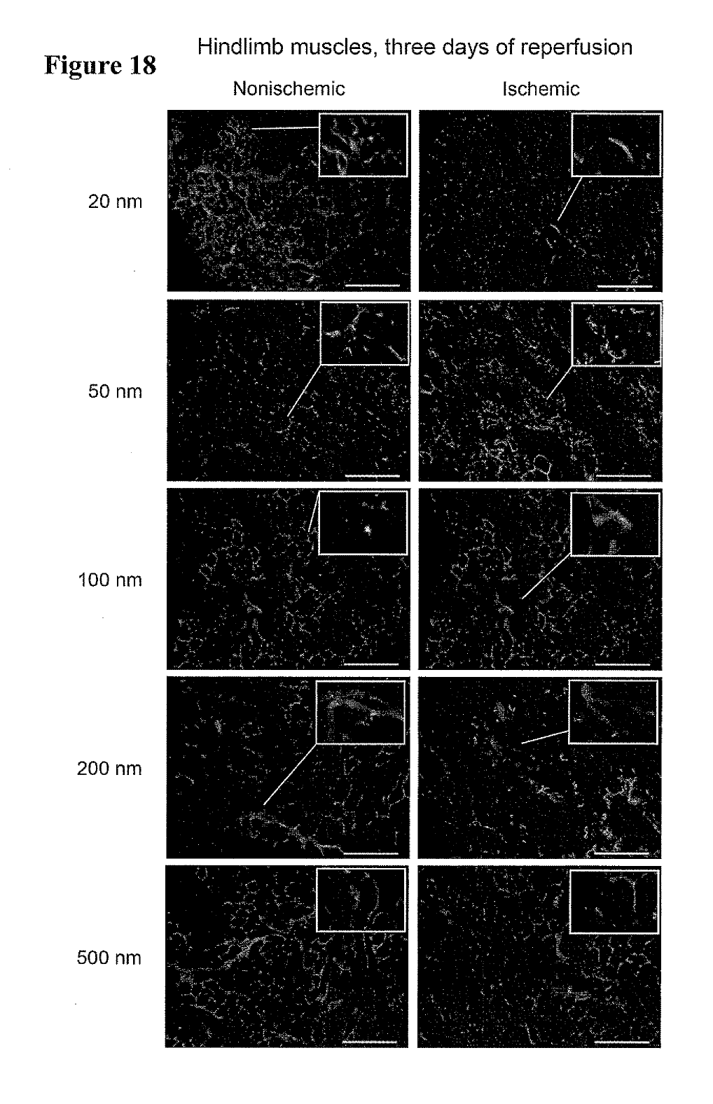

In contrast, local injuries may be not accompanied by the same heightened systemic response by the body as with systemic inflammation. To investigate whether changes in the microenvironmental conditions surrounding the diseased tissue altered the nanoparticle kinetics, a hindlimb ischemia-reperfusion model was performed as described previously²³ with some modifications. After femoral arterial ligation for 1 hour, the artery was allowed to reperfuse for 6 hours, 1 day, or 3 days (FIG. 15a). Ligation and reperfusion of the femoral artery was confirmed by measuring blood flow (FIG. 15b). At different time points during reperfusion, nanoparticles were injected, and the muscles were collected, prepared, and analyzed in the same manner as outlined above. After 6 hours of reperfusion, large nanoparticles were retained in the ischemic muscle to a greater extent than in the non-ischemic muscle (FIG. 5a and FIGS. 16 and 17). After 1 day, there was no difference in the distribution of nanoparticles larger than 100 nm between ischemic and nonischemic muscles. However, nanoparticles smaller than 100 nm were present in the ischemic leg, and this was also confirmed in tissue sections (FIG. 5b). After 3 days of reperfusion, there were no differences in distribution among the different sizes of nanoparticles (FIG. 18). Taking blood flow into consideration with the inflammation response, these factors may have caused the changes in the size-dependent biodistribution. During the inflammation stages of ischemia and reperfusion, the biodistribution of the nanoparticles differed. These findings suggest that the intervention time may be crucial, depending on the disease and the size of the drug nanocarriers. Drug nanocarriers larger than 100 nm have a retention time window of several hours after reperfusion, but smaller drug nanocarriers may require a 1 day period after reperfusion to achieve optimal effects.

We used poly(lactic-co-glycolic acid) nanoparticles (PLGA NPs) to confirm our results in a relevant clinical setting. PLGA NPs have been well-established and well-characterized in several drug delivery systems^{3,24,25}. Previous studies have demonstrated success in therapeutic treatments, and drug-containing PLGA NPs are also approved by the US Food and Drug Administration for clinical use. Using PLGA

NPs, the drug nanocarrier design considerations were tested with more relevant biomaterial than fluorescent polystyrene NPs, allowing us to generalize design principles for all drug nanoparticles, regardless of the material. We theorized that a PLGA NP drug delivery carrier smaller than 100 nm in diameter would result in greater retention in the muscle of a hindlimb ischemia-reperfusion model. We synthesized 80 and 300 nm PLGA NPs conjugated with quantum dots (PLGA-QD NPs; FIG. 5c), injected them into hindlimb ischemic mice subjected to a 1 day reperfusion, and analyzed the retention using IVIS imaging (FIG. 5d). Consistent with our initial size-dependent data, the in viva imaging of the mice in a supine position showed the presence of 80 nm PLGA-QD NPs on the surface near the skin at the ischemic region, but the 300 nm PLGA-QD NPs were absent (FIG. 5d). Ex vivo images of the muscle and subsequent quantification revealed that ischemia and reperfusion increased the retention of 80 nm PLGA-QD NPs compared with the 300 nm nanoparticles (FIG. 5d, e). This result was also consistent with our 1 day reperfusion data. Hindlimb ischemia and reperfusion induced local inflammatory responses, including vasodilation and increased blood flow. The 80 nm PLGA NPs were small enough to reperfuse into the muscle region of the inflamed hindlimb and escape renal reabsorption. In summary, we have validated the size-dependent biodistribution pattern of nanoparticles injected intravenously into mice and have confirmed that the alteration in the distribution pattern is caused by a physiologic change.

In the present study, the size-dependent biodistribution of nanoparticles ranging from 20 to 500 nm was systematically characterized in a mouse model. Our results indicate that most of the vital organs retained the nanoparticles in a size-dependent manner. Larger nanoparticles, particularly those with a diameter greater than 100 nm, were more likely to be distributed in the vital organs. Small nanoparticles, with a diameter of less than 100 nm, were mostly retained in the peripheral tissues or were excreted via the urine. Additionally, systemic inflammation and local hindlimb ischemia altered the biodistribution pattern to allow large nanoparticles to be retained in the vital organs and in the peripheral tissues. Our results were validated by the injection of a nanoparticles produced from an FDA approved material, PLGA. We consider that the comprehensive characterization of nanoparticle behavior in vivo presented in this study is important for nanomedicine design considerations. The conclusions drawn from our results should be taken into account when designing nanoparticles for intravenous drug delivery.

### REFERENCES

1. Anderson, D. G, Burdick, J. A. & Langer, R. Smart biomaterials. Science 305, 1923-1924 (2004).

2. Langer, R. & Tirrell, D. A. Designing materials for biology and medicine. Nature 428, 487-492 (2004).

3. Woodrow, K. A. et al. Intravaginal gene silencing using biodegradable polymer nanoparticles densely loaded with small-interfering RNA. Nat. Mater 8, 526-533 (2009).

4. Sengupta, S. et al. Temporal targeting of tumour cells and neovasculature with a nanoscale delivery system. Nature 436, 568-572 (2005).

5. Uygun, B. E. et al. Organ reengineering through development of a transplantable recellularized liver graft using decellularized liver matrix. Nat. Med. 16, 814-820 (2010).

6. Lv, S. et al. Designed biomaterials to mimic the mechanical properties of muscles. Nature 465, 69-73 (2010).

7. Soo Choi, H. et al. Renal clearance of quantum dots. Nat. Biotech. 25, 1165-1170 (2007).

8. Lee, S. et al. A near-infrared-fluorescence-quenched gold-nanoparticle imaging probe for in vivo drug screening and protease activity determination. Angew. Chem. Int. Edit. 47, 2804-2807 (2008).

9. Li, S. D. & Huang, L. Pharmacokinetics and biodistribution of nanoparticles. Mol. Pharm. 5, 496-504 (2008).

10. Choi, H. S. et al. Design considerations for tumour-targeted nanoparticles. Nat. Nanotechnol. 5, 42-47 (2010).

11. Nel, A. E. et al. Understanding biophysicochemical interactions at the nano-bio interface. Nat. Mater 8, 543-557 (2009).

12. Caldorera-Moore, M., Guimard, N., Shi, L. & Roy, K. Designer nanoparticles: incorporating size, shape and triggered release into nanoscale drug carriers. Expert Opin. Drug Deliv. 7, 479-495 (2010).

13. Mitragotri, S. & Lahann, J. Physical approaches to biomaterial design. Nat. Mater 8, 15-23 (2009).

14. He, C., Hu, Y., Yin, L., Tang, C. & Yin, C. Effects of particle size and surface charge on cellular uptake and biodistribution of polymeric nanoparticles. Biomaterials 31, 3657-3666 (2010).

15. Keren, S., Gheysens, 0., Levin, C. S. & Gambhir, S. S. A comparison between a time domain and continuous wave small animal optical imaging system. IEEE Trans. Med Imaging 27, 58-63 (2008).

16. Choi, C. H. J., Zuckerman, J. E., Webster, P. & Davis, M. E. Targeting kidney mesangium by nanoparticles of defined size. Proc. Natl. Acad. Sci. U.S.A. 108, 6656-6661 (2011).

17. Medzhitov, R. Origin and physiological roles of inflammation. Nature 454, 428-435 (2008).

18. Ashburner, J. et al. Computer-assisted imaging to assess brain structure in healthy and diseased brains. Lancet Neurol. 2, 79-88 (2003).

19. Bouwstra, J. A. & Ponec, M. The skin barrier in healthy and diseased state. B.B.A.-Biomembranes 1758, 2080-2095 (2006).

20. Venkataraman, C., Shankar, G., Sen, G & Bondada, S. Bacterial lipopolysaccharide induced B cell activation is mediated via a phosphatidylinositol 3-kinase dependent signaling pathway. Immunol. Lett. 69, 233-238 (1999).

21. Cinamon, G., Zachariah, M. A., Lam, 0. M., Foss, F. W. & Cyster, J. G. Follicular shuttling of marginal zone B cells facilitates antigen transport. Nat. Immunol. 9, 54-62 (2008).

22. Granucci, F. et al. Early events in dendritic cell maturation induced by LPS. Microbes and Infect. 1, 1079-1084 (1999).

23. Tang, Z. C. W., Liao, W.-Y., Tang, A. C. L., Tsai, S.-J. & Hsieh, P. C. H. The enhancement of endothelial cell therapy for angiogenesis in hindlimb ischemia using hyaluronan. Biomaterials 32, 75-86 (2011).

24. Liu, T. Y., Hu, S. H., Liu, D. M., Chen, S. Y. & Chen, I. W. Biomedical nanoparticle carriers with combined thermal and magnetic responses. Nano Today 4, 52-65 (2009).

25. Cheng, F.-Y. et al. Stabilizer-free poly(lactide-co-glycolide) nanoparticles for multimodal biomedical probes. Biomaterials 29, 2104-2112 (2008).

What is claimed is:

1. A method for screening the size of carrier for a subject in need, comprising:
(a) providing a series of labeled carriers which have different sizes;
(b) administering one of the series of carriers to a subject who suffers from a condition selected from organ dysfunction, inflammation, cancer formation or other injured or abnormal conditions;
(c) monitoring biodistribution of the carrier of step (b) in said subject;
(d) repeating steps (b) and (c) until all the series of carriers are administered and all the biodistribution of the series of carriers are monitored; and
(e) determining the size of carrier for said subject in accordance with the retention amount of the series of carriers in the tissue and/or organ affected by the condition of said subject.

2. The method according to claim 1, wherein the series of carriers are composed of an organic material, an inorganic material, or a metal material.

3. The method according to claim 1, wherein the series of carriers are nanoparticles having a size in the range of 0.1-1000 nm.

4. The method according to claim 1, wherein each of the series of carriers is fluorescence-labeled, radio-labeled, iron oxide-loaded, labeled by other materials or by methods for detection of the nanoparticles.

5. The method according to claim 1, wherein each of the series of carriers is administered by systemic intravascular, intramuscular or subcutaneous injection, oral intake, inhalation, or local skin, anal or vaginal administration.

6. The method according to claim 1, wherein the biodistribution of each carrier is monitored through in vivo, ex vivo or in vitro imaging system.

7. The method according to claim 1, wherein the organ affected by the condition is brain.

8. The method according to claim 7, wherein the size of carrier is in the range of 0.1-1000 nm.

9. The method according to claim 1, wherein the organ affected by the condition is skin.

10. The method according to claim 9, wherein the size of carrier is in the range of 0.1-1000 nm.

11. The method according to claim 1, wherein the tissue affected by the condition is muscle.

12. The method according to claim 11, wherein the size of carrier is in the range of 0.1-1000 nm.

13. The method according to Claim I, wherein the organ affected by the condition is liver or spleen.

14. The method according to claim 13, wherein the size of carrier is in the range of 0.1-1000 nm.

15. The method according to claim 1, wherein the organ affected by the condition is lung.

16. The method according to claim 15, wherein the size of carrier is in the range of 0.1-1000 nm.

17. The method according to claim 1, wherein the organ affected by the condition is kidney.

18. The method according to claim 17, wherein the size of carrier is in the range of 0.1-1000 nm.
